(19) United States
(12) Patent Application Publication
(10) Pub. No.: US 2006/0058243 A1
(43) Pub. Date: Mar. 16, 2006

(54) Methods and compositions for treating diabetes mellitis

(76) Inventors: Xiaozhuo Chen, Athens, OH (US); Yun Sheng Li, Athens, OH (US); Jing Li, Coral Springs, FL (US); Liu Fang, Vacaville, CA (US)

Correspondence Address: CALFEE HALTER & GRISWOLD, LLP, 800 SUPERIOR AVENUE, SUITE 1400, CLEVELAND, OH, 44114, US

(21) Appl. No.: 10/522,662

(22) PCT Filed: Jul. 24, 2002
(86) PCT No.: PCT/US02/23523

§ 371 Date: Sep. 9, 2005

(51) Int. Cl. A61K 31/7024

(52) U.S. Cl. 514/23; 514/25

(57) ABSTRACT

Methods for modulating diabetes, impaired glucose tolerance, gestational diabetes and glucose resistance in a mammal, particularly a human. In one embodiment the method comprises administering a gallotannin composition to a mammal in need of the same. The gallotannin composition comprises one or more select hydrolysable gallotannins. In another embodiment the method comprises administering a gallotannin variant composition comprising one ore more select gallotannin variant compounds to the subject. Methods of preventing or treating weight gain in a subject. The method comprises administering the gallotannin composition of the present invention, the gallotannin variant composition of the present invention, or a combination of the gallotannin composition of the present invention and the gallotannin variant composition of the present invention to the subject. The present invention also relates a gallotannin variant compound or a salt thereof, and a pharmaceutical composition comprising such compound or the salt thereof.

### FIELD OF THE INVENTION

The invention relates to methods and compositions for modulating diabetes mellitus and other disorders related to abnormal glucose and/or insulin levels in a mammalian subject. The present methods employ compositions that do not induce adipogenesis or hypoglycemia.

### BACKGROUND OF THE INVENTION

Diabetes mellitus, commonly called diabetes, refers to a disease process derived from multiple causative factors and characterized by elevated levels of plasma glucose, referred to as hyperglycemia. See, e.g., LeRoith, D. et al., (eds.), DIABETES MELLITUS (Lippincott-Raven Publishers, Philadelphia, Pa. U.S.A. 1996), and all references cited therein. According to the American Diabetes Association, diabetes mellitus is estimated to affect approximately 6% of the world population. Uncontrolled hyperglycemia is associated with increased and premature mortality due to an increased risk for microvascular and macrovascular diseases, including nephropathy, neuropathy, retinopathy, hypertension, cerebrovascular disease and coronary heart disease. Therefore, control of glucose homeostasis is an important approach for the treatment of diabetes.

There are two major forms of diabetes: Type 1 diabetes (formerly referred to as insulin-dependent diabetes or IDDM); and Type 2 diabetes (formerly referred to as noninsulin dependent diabetes or NIDDM). Type 1 diabetes is the result of an absolute deficiency of insulin, the hormone which regulates glucose utilization. This insulin deficiency is usually characterized by β-cell destruction within the Islets of Langerhans in the pancreas and absolute insulin deficiency. Type 2 diabetes is a disease characterized by insulin resistance accompanied by relative, rather than absolute, insulin deficiency. Type 2 diabetes can range from predominant insulin resistance with relative insulin deficiency to predominant insulin deficiency with some insulin resistance. Insulin resistance is the diminished ability of insulin to exert its biological action across a broad range of concentrations. In insulin resistant individuals the body secretes abnormally high amounts of insulin to compensate for this defect. When inadequate amounts of insulin are present to compensate for insulin resistance and adequately control glucose, a state of impaired glucose tolerance develops. In a significant number of individuals, insulin secretion declines further and the plasma glucose level rises, resulting in the clinical state of diabetes.

The majority of Type 2 diabetic patients are treated either with hypoglycemic agents which act by stimulating release of insulin from beta cells, or with agents that enhance the tissue sensitivity of the patients towards insulin, or with insulin. Sulfonylureas are examples of agents that stimulate release of insulin from beta cells. Among the agents applied to enhance tissue sensitivity towards insulin, metformin is a representative example. Even though sulfonylureas are widely used in the treatment of type II diabetes, this therapy is, in most instances, not satisfactory. In a large number of type II diabetic patients sulfonylureas do not suffice to normalize blood sugar levels and the patients are, therefore, at high risk for acquiring diabetic complications. Also, many patients gradually lose the ability to respond to treatment with sulfonylureas and are, thus, gradually forced into insulin treatment. This shift of patients from oral hypoglycemic agents to insulin therapy is usually ascribed to exhaustion of the pancreatic 0 cells in type II diabetic patients.

Insulin stimulates glucose uptake by skeletal muscle and adipose tissues primarily through translocation of the glucose transporter 4 (GLUT4) from the intracellular storage sites of the cell surface (Saltiel, A. R. & Kahn, C. R. (2001) Nature 414:799-806; Saltiel, A. & Pessin, J. E. (2002) Trends in Cell Biol. 12:65-71; White, M. F. (1998) Mol. Cell. Biochem. 182:3-11). In response to insulin, a fraction of GLUT4 present in intracellular membranes is redistributed to the plasma membrane resulting in an increase of GLUT4 on the cell surface and enhanced glucose uptake by these cells. GLUT4 translocation is primarily mediated through the insulin receptor (IR).

In addition to glucose transport, insulin is intimately involved in adipogenesis, a process which involves proliferation of preadipocytes (pre-fat cells) and differentiation of preadipocytes into adipocytes (fat cells) with accumulation of fat in adipocytes. Studies with the adipocyte cell line 3T3-L1 suggest that the role insulin plays in adipogenesis is primarily mitotic (43). Before differentiation, 3T3-L1 cells are fibroblast-like preadipocytes that contain more IGF-1 receptors than IR. In vitro, adipogenesis of preadipocytes can be triggered by a commonly used differentiation-inducing cocktail, MDI, which consists of an agent methylisobutylxanthine (MIX) that elevates cAmp; a glucocorticoid, dexamethasone (DEX); and insulin (or IGF-1) that interacts with the IGF-1 receptors on the preadipocytes (Tong, Q., Hotamisligil, G. S. (2001) Rev. in Endoc. & Metabolic Disorders. 2:349-355; Rosen, E. D., et al. (2000) Genes Dev. 14:1293-1307). When treated with MDI, confluent preadipocytes re-enter the cell cycle and undergo approximately two rounds of mitosis (Modan-Moses, D., et al. (1998) Biochem. J. 333:825-831; Tong, Q., Hotamisligil, G. S. (2001) Rev. in Endoc. & Metabolic Disorders. 2:349-355; Rosen, E. D., et al. (2000) Genes Dev. 14:1293-1307), a process commonly referred to as clonal expansion. Following clonal expansion, the preadipocytes exit the cell cycle and begin to differentiate into adipocytes by expressing adipocyte genes including C/EBP-α, β, δ, and PPAR-γ.

As a result of its adipogenic effect, insulin has the undesirable effect of promoting obesity in patients with type 2 diabetes. (See, Moller, D. E. (2001) Nature 414:821-827) Unfortunately, other anti-diabetic drugs which are currently being used to stimulate glucose transport in patients with type 2 diabetes also possess adipogenic activity. Thus while current drug therapy may provide reduction in blood sugar, it often promotes obesity. Accordingly, it is highly desirable to develop a new generation of anti-diabetic drugs that correct hyperglycemia without generating concomitant adipogenic side effects. Compounds that induce glucose uptake in a diabetic patient without causing hypoglycemia are also desirable.

### SUMMARY OF THE INVENTION

The present invention provides methods for modulating diabetes, impaired glucose tolerance, gestational diabetes and glucose resistance in a mammal, particularly a human. In one embodiment the method comprises administering a composition, referred to hereinafter as a “gallotannin composition” to a mammal in need of the same. The gallotannin composition is substantially pure and comprises one or more hydrolysable gallotannins selected from the group consisting of 1,2,3,4-tetra-O-galloyl-α-D-glucose, 1,2,3,6-tetra-O-galloyl-α-D-glucose, 1,3,4,6-tetra-O-galloyl-α-D-glucose, 1,2,3,4,6-penta-O-galloyl-α-D-glucose, 1,2,3,4,6-penta-O-galloyl-β-D-glucose, 1,2,3,4,6-hexa-galloyl-α-D-glucose, 1,2,3,4,6-hexa-O-galloyl-β-D-glucose, 1,2,3,4,6-hepta-O-galloyl-α-D-glucose, 1,2,3,4,6-hepta-O-galloyl-β-D-glucose, 1,2,3,4,6-octa-O-galloyl-α-D-glucose, 1,2,3,4,6-octa-O-galloyl-β-D-glucose, 1,2,3,4,6-nona-O-galloyl-β-D-glucose, 1,2,3,4,6-nona-O-galloyl-β-D-glucose, 1,2,3,4,6-deca-O-galloyl-α-D-glucose, and 1,2,3,4,6-deca-O-galloyl-β-D-glucose. As used herein the term “substantially pure” means that the gallotannin composition comprises at least 95% by dry weight of one or a combination of the listed gallotannins and less than 5% by dry weight of one or more of the following compounds: mono-O-galloyl-β-D-glucose, di-O-galloyl-β-D-glucose, tri-O-galloyl-β-D-glucose, tetra-O-galloyl-β-D-glucose, unadeca-O-galloyl-β-D-glucose, dodeca-O-galloyl-β-D-glucose or mixtures thereof.

In another embodiment the method comprises administering a composition referred to hereinafter as a “gallotannin variant composition” to the subject. The gallotannin variant composition comprises one or more gallotanin variant compounds or salts thereof. The gallotannin variant compounds have the following structure:
R—X-A(ₙ)-X-A(_{q})-X-A(_{z}),
wherein R is selected from the group consisting of D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose, x-D-Xylose, α-D Lyxose, β-D Lyxose, α-D Arabinose, β-D Arabinose, α-D Ribose, β-D Ribose, D-Trehalose, D-Maltose, D-Cellobiose, myo-Inositol, D-glucitol,
X is an ester or ether linkage,
A is a trihydroxybenzoic acid selected from the group consisting of 3,4,5-trihydroxybenzoic acid, 2,3,4-trihydroxybenzoic acid 2,4,6-trihydroxybenzoic acid, or a dihydroxybenzoic acid selected from the group consisting of 2,3-dihydroxybenzoic acid, 2,4-dihydroxybenzoic acid, 3,4-dihydroxybenzoic acid, or a monohydroxybenzoic acids selected from the group consisting of 3-hydroxybenzoic acid and 4-hydroxybenzoic acid,
wherein n is 5, q is 0, 1, 2, 3, 4, or 5, and z is 0 when R is D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose;
wherein n is 4, q is 0, 1, 2, 3, or 4, and z is 0, 1, or 2 when R is α-D-Xylose, α-D Lyxose, β-D Lyxose, α-D Arabinose, β-D Arabinose, α-D Ribose, β-D Ribose;
wherein n is 6, q is 0, 1, 2, 3, 4, 5, or 6, and z is 0 when R is D-Glucitol or myo-Inositol, and
wherein n is 8, q is 0, 1, 2, 3, 4, 5, 6, 7, or 8, and z is 0 when R is D-Trehalose, D-Maltose, or D-Cellobiose.

Each of the compounds in the gallotannin variant composition has a structure other than the structure of tetra-O-galloyl-β-D-glucose, 1,2,3,4,6-penta-O-galloyl-β-D-glucose, 1,2,3,4,6hexa-O-galloyl-β-D-glucose, 1,2,3,4,6-hepta-O-galloyl-β-D-glucose, 1,2,3,4,6-octa-O-galloyl-β-D-glucose, 1,2,3,4,6-nona-O-galloyl-β-D-glucose, and 1,2,3,4,6-deca-O-galloyl-β-D-glucose.

In a third embodiment the method comprises administering a combination of the gallotannin composition of the present invention and the gallotannin variant composition of the present invention to the patient.

The present invention also provides methods of preventing or treating weight gain in a subject. The method comprises administering the gallotannin composition of the present invention, the gallotannin variant composition of the present invention, or a combination of the gallotannin composition of the present invention and the gallotannin variant composition of the present invention to the subject.

The present invention also provides methods of inhibiting differentiation of preadipocytes into adipocytes. The methods comprise contacting the pre-adipoctyes with the natural gallotannin composition of the present invention, the gallotannin variant composition of the present invention, or a combination of the natural gallotannin composition of the present invention and the gallotannin variant composition of the present invention. The pre-adipoctyes may be in culture or in a mammalian subject.

The present invention also relates to the present gallotannin variant composition, a compound having the structure, R—X-A(ₙ)-X-A(_{q})-X-A(_{z}), or a salt thereof, and a pharmaceutical composition comprising such compound or the salt thereof.

### BRIEF DESCRIPTION OF THE FIGURES

FIG. 1. Structure of penta-O-galloyl-D-glucose (PGG).

PGG consists of a glucose core that is covalently linked to five gallic acids through ester bonds. With two possible configurations at carbon 1(*) of glucose, two anomers of PGG exist. Computer simulated PGG conformations indicate that α-PGG may be more symmetrical (thus less polar) than β-PGG.

FIG. 2. Glucose transport stimulatory activity and anti-adipogenic activity of PGG.

PGG, either as a mixture or as single isomer (α or β), is added to either adipocytes (A) or preadipocytes (B). GTS activity (A) and AD activity (B) of PGG are measured by ³H-glucose uptake. GLUT4 is not expressed in preadipocytes, so glucose uptake can be used as an indirect measurement of adipocyte differentiation.

FIG. 3. PGG binds IR with Kd of ˜10⁻⁵M.

3T3-L1 adipocytes in 24-well plates were incubated overnight at 4 C with increasing concentrations of cold insulin in the presence of 8 pM ¹²⁵I-insulin or with increasing concentrations of cold PGG in the presence of 20 μM ¹⁴C-PGG, respectively, and then measured for cell-bound ¹²⁵I-insulin or ¹⁴C-PGG after removal of unbound isotope.

FIG. 4. PGG does not displace insulin binding to its receptor IR in 3T3-L1 adipocytes.

3T3-L1 adipocytes were incubated overnight at 4° C. with increasing concentrations of ¹⁴C-PGG in the presence of 8 pM ¹²⁵I-labeled insulin, and then counted for cell-bound I²⁵1-insulin and ¹⁴C-PGG. Near-constant insulin counts between 0.1 to 20 μM of PGG indicate PGG is unable to displace insulin from IR at this concentration range. Insulin binding increased at higher PGG concentrations.

FIG. 5. Protein binding selectivity of PGG.

A fixed quantity of ¹²⁵I BSA was incubated with PGG in the presence of variable amount of either cold gelatin, or BSA, or ovalbumin. Bound was separated from free by precipitation and was expressed as the % bound in the absence of competitor. The three competition curves indicate that PGG selectively binds different proteins with significantly different affinities.

FIG. 6. Three insulin-signaling pathway-specific inhibitors also abolish PGG-induced glucose transport in 3T3-L1 adipocytes.

Adipocytes were induced by insulin or PGG in the presence or absence of different inhibitors. The glucose transport activity of the treated cells was measured by ³H glucose taken up by the cells. HNMPA-(AM)₃ inhibits IR Tyr kinase activity, Cytochalasin B inhibits GLUT4, Wortmannin inhibits PI-3K, respectively.

FIG. 7. PGG induces phosphorylation of Akt in 3T3-L1 adipocytes.

Differentially treated adipocytes were lysed, the cell proteins analyzed by SDS-PAGE. I=untreated; 2=insulin, 3=PGG, 15 μM; 4=PGG, 30 μM; M=Protein size markers.

FIG. 8. Oil Red 0 staining of 3T3-L1 cells induced by MDI or insulin plus 30 μM PGG.

Ten days after induction, the cells were stained with Oil Red 0 and photographed at magnification X200. Only those cells that contain fat vesicles (triglyceride) can be stained.

FIG. 9. Northern blot analysis of PPARγ and C/EBPα gene expression in 3T3-L1 preadipocytes. Differentially treated preadipocytes were analyzed for their mRNA expression at various times post treatment. Lane 1: No treatment; Lane 2=MDI; Lane 3=30 μM PGG+MDI; Lane 4=30 μM of PGG alone. Times=times post treatment when mRNA was isolated.

FIG. 10. Clonal expansion in α-PGG and β-PGG treated 3T3-L1 preadipocytes.

Preadipocytes were induced to undergo clonal expansion by MDI, or α-PGG, or β-PGG in the presence of MDI. 24 or 48 hrs after induction, the media was removed and the cells were lysed. The lactose dehydrogenase (LDH) activity of the cell lysates was measured by LDH kit. Cell growth media were also collected and their LDH activity from dead cells was also measured (not shown on this graph). LDH activity is a constant per cell in a given cell type, the LDH activity measured is proportional to the number of cells in the samples.

FIG. 11. Effect of PGG on blood glucose levels in db/db and ob/ob mice. Various doses of α-PGG were orally delivered without glucose to db/db mice(A) or with glucose to ob/ob mice (B) mice. At different times post the delivery, glucose was determined in samples from tail blood.

FIG. 12. PGG protects ob/ob mice from hyperglycemia immediately after glucose challenge and hypoglycemia several days after the challenge. The ob/ob mice underwent a glucose tolerance test as shown in FIG. 111B. At various time points after the glucose challenge, blood glucose levels were measured in tail blood.

FIG. 13. Chemical structure of select gallotannin variants. G represents a trihydroxybenzoic acid

FIG. 14. Effect of PGG on plasma insulin levels in ob/ob mice.

FIG. 15. Effect of PGG on blood glucose levels in mice with normal blood glucose levels.

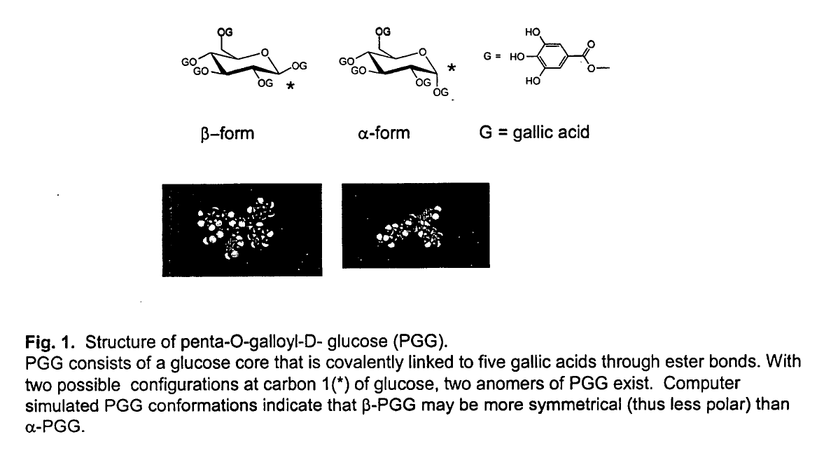

### DETAILED DESCRIPTION OF THE INVENTION

Definitions

The term “diabetes mellitus” or “diabetes” means a disease or condition that is generally characterized by metabolic defects in production and utilization of glucose which result in the failure to maintain appropriate blood sugar levels in the body. The result of these defects is elevated blood glucose, referred to as “hyperglycemia.” Two major forms of diabetes are Type 1 diabetes and Type 2 diabetes. As described above, Type 1 diabetes is generally the result of an absolute deficiency of insulin, the hormone which regulates glucose utilization. Type 2 diabetes often occurs in the face of normal, or even elevated levels of insulin and can result from the inability of tissues to respond appropriately to insulin. Most Type 2 diabetic patients are insulin resistant and have a relative deficiency of insulin, in that insulin secretion can not compensate for the resistance of peripheral tissues to respond to insulin. In addition, many Type 2 diabetics are obese. Other types of disorders of glucose homeostasis include impaired glucose tolerance, which is a metabolic stage intermediate between normal glucose homeostasis and diabetes, and gestational diabetes mellitus, which is glucose intolerance in pregnancy in women with no previous history of Type 1 or Type 2 diabetes.

The guidelines for diagnosis for Type 2 diabetes, impaired glucose tolerance, and gestational diabetes have been outlined by the American Diabetes Association (see, e.g., The Expert Committee on the Diagnosis and Classification of Diabetes Mellitus, Diabetes Care, (1999) Vol 2 (Suppl 1): S5-19).

The term “symptom” of diabetes, includes, but is not limited to, polyuria, polydipsia, and polyphagia, hyperinsulinemia, and hyperglycemia as used herein, incorporating their common usage. For example, “polyuria” means the passage of a large volume of urine during a given period; “polydipsia” means chronic, excessive thirst; “polyphagia” means excessive eating, and hyperinsulinemia means elevated blood levels of insulin. Other symptoms of diabetes include, for example, increased susceptibility to certain infections (especially fungal and staphylococcal infections), nausea, and ketoacidosis (enhanced production of ketone bodies in the blood).

The term “complication” of diabetes includes, but is not limited to, microvascular complications and macrovascular complications. Microvascular complications are those complications which generally result in small blood vessel damage. These complications include, e.g., retinopathy (the impairment or loss of vision due to blood vessel damage in the eyes); neuropathy (nerve damage and foot problems due to blood vessel damage to the nervous system); and nephropathy (kidney disease due to blood vessel damage in the kidneys). Macrovascular complications are those complications which generally result from large blood vessel damage. These complications include, e.g., cardiovascular disease and peripheral vascular disease. Cardiovascular disease refers to diseases of blood vessels of the heart. See e.g., Kaplan, R. M., et al., “Cardiovascular diseases” in HEALTH AND HUMAN BEHAVIOR, pp. 206-242 (McGraw-Hill, New York 1993). Cardiovascular disease is generally one of several forms, including, e.g., hypertension (also referred to as high blood pressure), coronary heart disease, stroke, and rheumatic heart disease. Peripheral vascular disease refers to diseases of any of the blood vessels outside of the heart. It is often a narrowing of the blood vessels that carry blood to leg and arm muscles.

“Hydrolysable gallotannin” as used herein refers to a galloyl glucose compound which is an ester of glucose with one or more trihydroxybenzene carboxylic acids. The substantially pure hydrolysable gallotannin composition of the present invention comprises one or more alpha or beta anomers of hydrolysable gallotannins having 5, 6, 7, 8, 9, or 10 galloyl groups, or pharmaceutically acceptable salts thereof. The hexa, hepta, octa, nona, and deca forms of the hydrolysable gallotannins each have an initial set of galloyl groups attached to carbons 1, 2, 3, 4, and 6 of the glucose core and a second set of galloyl groups comprising from 1 to 5 additional galloyl groups, respectively. The galloyl groups of the second set are attached to separate galloyl groups in the first set. The substantially pure gallotannin composition of the present invention may also comprise 1,2,3,4-tetra-O-galloyl-α-D-glucose, 1,2,3,6-tetra-O-galloyl-α-D-glucose, 1,3,4,6-tetra-O-galloyl-α-D-glucose.

The beta anomers of hydrolysable gallotannins are found in many plant-based foods including common fruits (berries, bananas, grapes, apples); grains (barley, sorghum); plant-derived beverages such as tea and wine. Typically, the beta anomers of hydrolysable gallotannins are also found in tannic acid mixtures which are commercially available. Commercial tannic acid mixtures also comprise varying amounts of methyl galloyl and galloyl glucose compounds that comprise 1, 2, 3, 11 and 12 galloyl groups. The substantially pure hydrolysable gallotannin compositions of the present invention comprise less than 5% by dry weight, preferably less than 3% by dry weight, more preferably less than 1% by dry weight, of one or a mixture of the following compounds: mono-O-galloyl-β-D-glucose, di-O-galloyl-β-D-glucose, tri-O-galloyl-β-D-glucose, tetra-O-galloyl-β-D-glucose, unadeca-O-galloyl-β-D-glucose, dodeca-O-galloyl-β-D-glucose or mixtures thereof. Thus, the substantially pure hydrolysable gallotannin compositions that are used in the present methods are different from commercially available mixtures of gallotannins.

“Gallotannin Variant” as used herein refers to a compound that is similar to but not identical in structure to tetra-O-galloyl-β-D-glucose, 1,2,3,4,6-penta-O-galloyl-β-D-glucose, hexa-O-galloyl-β-D-glucose, hepta-O-galloyl-β-D-glucose, octa-O-galloyl-β-D-glucose, nona-O-galloyl-β-D-glucose, deca-O-galloyl-β-D-glucose, unadeca-O-galloyl-β-D-glucose, and dodeca-O-galloyl-β-D-glucose.

The variant has the following structure:
R—X-A(ₙ)-X-A(_{q})-X-A(_{z}),
wherein R is selected from the group consisting of D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose, α-D-Xylose, α-D Lyxose, β-D Lyxose, α-D Arabinose, β-D Arabinose, α-D Ribose, β-D Ribose, D-Trehalose, D-Maltose, D-Cellobiose, myo-Inositol, D-glucitol,
X is an ester or ether linkage,
A is a trihydroxybenzoic acid selected from the group consisting of 3,4,5-trihydroxybenzoic acid, 2,3,4-trihydroxybenzoic acid 2,4,6-trihydroxybenzoic acid, or a dihydroxybenzoic acid selected from the group consisting of 2,3-dihydroxybenzoic acid, 2,4-dihydroxybenzoic acid, 3,4-dihydroxybenzoic acid, or a monohydroxybenzoic acids selected from the group consisting of 3-hydroxybenzoic acid and 4-hydroxybenzoic acid,
wherein n is 5, q is 0, 1, 2, 3, 4, or 5, and z is 0 when R is D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose;
wherein n is 4, q is 0, 1, 2, 3, or 4, and z is 0, 1, or 2 when R is α-D-Xylose, α-D Lyxose, β-D Lyxose, α-D Arabinose, β-D Arabinose, α-D Ribose, β-D Ribose;
wherein n is 6, q is 0, 1, 2, 3, 4, 5, or 6, and z is 0 when R is D-Glucitol or myo-Inositol, and
wherein n is 8, q is 0, 1, 2, 3, 4, 5, 6, 7, or 8, and z is 0 when R is D-Trehalose, D-Maltose, or D-Cellobiose.

“Adipocytes” as used herein refers to fat cells. Morphologically, adipocytes are round-shaped, triglyceride (fat) vesicle-containing cells. Biochemically, adipocytes express high levels of insulin receptor on their cell surface and exhibit a highly active insulin-mediated glucose transport signaling pathway involving glucose transporter 4 (GLUT4). In vivo, adipocytes are involved in the synthesis and storage of fat (triglyceride) and glucose metabolism (uptake of glucose from blood and conversion of glucose into fat).

“Preadipocytes” as used herein refers to adipocyte precursor cells that, under the action of hormones such as insulin and glucocorticoid, divide and differentiate into adipocytes. Morphologically, preadipocytes are fibroblast-looking (thin, and spindle-shaped) and devoid of triglyceride (fat) vesicles in their cytoplasm. As compared to adipocytes, preadipocytes contain low levels of insulin receptor and relatively high levels of insulin-like growth factor 1 (IGF-1) receptors for receiving mitogenic and differentiating signals. Without induction or full differentiation, preadipocytes do not express GLUT4 or other differentiation related genes such as PPAR-γ, C/EBP-α or C/EBP-γ. The intracellular glucose transport activity of preadipocytes is lower than that of adipoctyes.

“Adipogenesis” as used herein refers to the process by which preadipocytes divide and differentiatiate into adipocytes.

“Lipogenesis” as used herein refers to the process by which fat is synthesized and accumulated in adipocytes.

The term “mammal” includes, without limitation, humans, domestic animals (e.g., dogs or cats), farm animals (cows, horses, or pigs), monkeys, rabbits, mice, and laboratory animals.

In one aspect, the present invention provides methods for stimulating uptake of glucose in the cells of a mammal, particularly a mammal with diabetes, impaired glucose intolerance, insulin resistance or gestational diabetes. In another aspect, the present invention provides methods of inhibiting differentiation of pre-adipocytes to adipocytes in a mammal, particular a mammal that is obese, overweight, or who is exhibiting symptoms of diabetes mellitus, glucose intolerance, or gestational diabetes. The present methods are based in part on inventors' discovery that certain hydrolysable gallotannins and certain gallotannin variants are able to stimulate glucose transport into adipocytes and to inhibit differentiation of pre-adipocytes into adipoctyes. The present methods are also based in part on inventors' discovery that certain hydrolysable gallotannins lower blood glucose levels and blood insulin levels in mammals. The present methods are also based, at least in part, on inventors' discovery they certain hydrolysable gallotannins do not cause hypoglycemia. Accordingly, the present methods are useful for treating or preventing diabetes, impaired glucose tolerance, insulin resistance and gestational diabetes in a mammal.

In one embodiment, the present method for treating or preventing diabetes, impaired glucose tolerance, insulin resistance and gestational diabetes in a mammal comprises administering a therapeutically effective amount of a substantially pure hydrolysable gallotannin composition to the mammal. The substantially pure gallotannin composition comprises one or more hydrolysable gallotannins selected from the group consisting of 1,2,3,4-tetra-O-galloyl-α-D-glucose, 1,2,3,6-tetra-O-galloyl-α-D-glucose, 1,3,4,6-tetra-O-galloyl-α-D-glucose, 1,2,3,4,6-penta-O-galloyl-α-D-glucose, 1,2,3,4,6-penta-O-galloyl-β-D-glucose, 1,2,3,4,6-hexa-galloyl-α-D-glucose, 1,2,3,4,6-hexa-O-galloyl-β-D-glucose, 1,2,3,4,6-hepta-O-galloyl-α-D-glucose, 1,2,3,4,6-hepta-O-galloyl-β-D-glucose, 1,2,3,4,6-octa-O-galloyl-α-D-glucose, 1,2,3,4,6-octa-O-galloyl-β-D-glucose, 1,2,3,4,6-nona-O-galloyl-α-D-glucose, 1,2,3,4,6-nona-O-galloyl-β-D-glucose, 1,2,3,4,6-deca-O-galloyl-α-D-glucose, and 1,2,3,4,6-deca-O-galloyl-β-D-glucose, or a pharmaceutically acceptable salt of the hydrolysable gallotannins. The substantially pure hydrolysable gallotannin composition comprises less than 5% by dry weight of one or more of the following compounds: mono-O-galloyl-β-D-glucose, di-O-galloyl-β-D-glucose, tri-O-galloyl-β-D-glucose, tetra-O-galloyl-β-D-glucose, unadeca-O-galloyl-β-D-glucose, dodeca-O-galloyl-β-D-glucose or mixtures thereof.

In another embodiment, the method for treating or preventing diabetes, impaired glucose tolerance, insulin resistance and gestational diabetes in a mammal comprises administering a therapeutically effective amount of a gallotannin variant composition comprising one or more gallotannin variant compounds to the patient. In a further embodiment, the method comprises administering both a substantially pure hydrolysable gallotannin composition of the present invention and a gallotannin variant composition to the patient.

Optionally, other agents which are used to treat or prevent diabetes, including insulin, Sulfonylureas, Meglitinides, biguanides (Glucophage or Metformin), Thiazolidinedione (TZDs), and alpha-glucosidase inhibitors, are administered to the mammal in combination with the present hydrolysable gallotannin or gallotannin variant composition. For those mammals with Type I diabetes mellitus, it is preferred that insulin be administered in combination with the gallotannin composition and/or the gallotannin variant composition.

Subjects

The present methods are useful for treating mammals who have been diagnosed as having diabetes, gestational diabetes, insulin resistance or impaired glucose tolerance. The present methods are also useful for treating mammals exhibiting symptoms of diabetes, gestational diabetes, insulin resistance or impaired glucose tolerance, or mammals that have a genetic predisposition to diabetes, gestational diabetes, insulin resistance or impaired glucose tolerance. The present methods are also useful for preventing or treating weight gain in a subject, particularly in subjects who are obese or overweight.

Modes of Administration

The present compositions are administered to the subject by injection, including subcutaneous, parenteral, and intravenous injection, or by oral administration. Because of its ease of administration, the preferred route of administration is oral administration.

Formulations

Gallotannin and gallotannin variant compositions for use in accordance with the present methods are formulated into pharmaceutical compositions using conventional methods. Such pharmaceutical preparations comprise one or more hydrolysable gallotannins of the present invention and/or one or more gallotannin variant compounds of the present invention. Optionally the pharmaceutical composition further comprises a pharmaceutically acceptable carrier or diluent. The term “pharmaceutically acceptable” means a non-toxic material that does not interfere with the effectiveness of the gallotannin or gallotannin variant composition. Many such carriers are routinely used and can be identified by reference to pharmaceutical texts. The characteristics of the carrier will depend on the route of administration and particular compound or combination of compounds in the composition. Preparation of such formulations is within the level of skill in the art. The preparation may further contain other agents that either enhance the activity of the gallotannin or gallotannin variant or complement its activity. The preparation may further comprise fillers, salts, buffers, stabilizers, solubilizers, and other materials well known in the art.

The pharmaceutical composition may conveniently be presented in unit doesage form and may be prepared by any of the methods well known in the art of pharmacy. The term “unit dosage” means a predetermined amount of the gallotannin or gallotannin variant composition sufficient to be effective in treating the target disease or disorder. All methods include the step of bringing the gallotannin composition the gallotannin variant composition or both into contact with the carrier or diluent and any other optional accessory ingredients.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate or talc); disintegrants (e.g., potato starch or sodium starch glycolate); or wetting agents (e.g., sodium lauryl sulphate). The tablets can be coated by methods well known in the art. Liquid preparations for oral administration can take the form of, for example, aqueous solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydrobenzoates or sorbic acid). The preparations can also contain buffer salts, flavoring, coloring and sweetening agents as appropriate. The preparations can also take the form of nutritional formulas. Preparations for oral administration can be formulated to give controlled release of the gallotannin composition. Because of the presence of high levels of proline containing proteins in the saliva, it is expected that the preferred oral formulation will be in the form of a capsule which comprises a coating to protect the gallotannin composition from interacting with the saliva

The gallotannin and gallotannin variant compositions can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form, tablets or capsules for constitution with a suitable vehicle, e.g., sterile pyrogen-free water, before use.

The gallotannin and gallotannin variant compositions can also be formulated in rectal compositions such as suppositories or retention enemas, e.g., containing conventional suppository bases such as cocoa butter, other glycerides or carbowax.

In addition to the formulations described previously, the gallotannin and gallotannin variant compositions can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### Dosage

The gallotannin or gallotannin variant composition is administered to the subject in a therapeutically effective amount. As used herein, the term “therapeutically effective amount” means the total amount that is sufficient to show a meaningful benefit, e.g., reduction of hyperglycemia (reduction of blood glucose level), reduction of hyperinsulinemia (reduction of blood insulin level), improvement in glucose tolerance, prevention of weight gain and weight loss. The dosages of gallotannin compostion or gallotannin variant composition needed to obtain a meaningful result can be determined in view of this disclosure by one of ordinary skill in the art by running routine trials with appropriate controls. Comparison of the appropriate treatment groups to the controls will indicate whether a particular dosage is effective at reducing the subject's blood glucose levels or inhibiting adipogenesis.

The amount of the gallotannin compositon required will depend upon the nature and severity of the condition being treated, and on the nature of prior treatments which the subject has undergone. Ultimately, the dosage will be determined using clinical trials. Initially, the clinician will administer doses that have been derived from animal studies. An effective amount can be achieved by one administration of the composition. Alternatively, an effective amount is achieved by multiple administration of the composition to the subject. In vitro, the biologically effective amount, i.e., the amount sufficient to induce glucose uptake, is administered in two-fold increments, to determine the full range of activity. The efficacy of oral, subcutaneous and intravenous administration is determined in clinical studies. Although a single administration of the gallotannin composition may be beneficial, it is expected that multiple doses will be preferred.

### Methods of Determining Dosages for Stimulating Glucose Uptake in Cells

Glucose uptake activity in cells may be analyzed by measuring the uptake of 2-deoxy-D-[³H] glucose using a standard assay. Confluent 3T3-L1 adipocytes grown in 12-well plates are washed twice with serum-free DMEM and incubated with 1 mL of the same medium at 37° C. for 2 h. The cells are washed 3 times with Krebs-Ringer-Hepes (KRP) buffer and incubated with 0.9 mL KRP buffer at 37° C. for 30 min. Insulin (positive control) or the gallotannin or gallotannin variant (experimentals) are then added at pre-determined concentrations and adipocytes are incubated at 37° C. for 15 min. Glucose uptake is initiated by addition of 0.1 mL KRP buffer and 37 MBq/L 2-deoxy-D-[³H] glucose and 1 mmol/L glucose as final concentrations. After 10 min, glucose uptake is terminated by washing the cells 3 times with cold PBS. The cells are lysed with 0.7 mL of 1% Triton X-100 at 37° C. for 20 min. The radioactivity retained by the cell lysates is determined by a scintillation counter. The dosage that induces the maximal glucose uptake can be selected among the experimental samples.

Methods of Determining Dosages for Stimulating Glucose Uptake in Animals

Male db/db (leptin receptor deficient) mice of 8 weeks of age may be used to determine in vivo dosages for simulating glucose uptake The mice are divided into three to four groups depending upon how many dosages are analyzed. Ten μl of a test solution with pre-determined concentrations of the test gallotannin composition is orally administered to the test mice. The negative control mice receive the same amount of water. After the administration, blood is collected from the mouse tail at various times post oral administration. The blood glucose level of a mouse at a given time post administration is measured by applying six μl of blood on a One Touch Basic Complete Diabetes Monitoring System (from Lifescan). The effective dosage range and the optimal dosage can be determined by comparison of the reduction of blood glucose levels by different dosages relative to the glucose level of the negative (water) control group.

Inhibition of Adipogenesis and Weight Gain

In another aspect, the present invention provides methods for inhibiting differentiation of preadipocytes into adipocytes. The adipocytes may be in culture or in a mammalian subject. In one embodiment, the method comprises contacting the preadipocytes with a biologically effective amount of hydrolysable gallotannin composition. The gallotannin composition is substantially pure and comprises The gallotannin composition comprises one or more hydrolysable gallotannins selected from the group consisting of 1,2,3,4-tetra-O-galloyl-α-D-glucose, 1,2,3,6-tetra-O-galloyl-α-D-glucose, 1,3,4,6-tetra-O-galloyl-α-D-glucose, 1,2,3,4,6-penta-O-galloyl-α-D-glucose, 1,2,3,4,6-penta-O-galloyl-α-D-glucose, 1,2,3,4,6-hexa-galloyl-α-D-glucose, 1,2,3,4,6-hexa-O-galloyl-β-D-glucose, 1,2,3,4,6-hepta-O-galloyl-α-D-glucose, 1,2,3,4,6-hepta-O-galloyl-β-D-glucose, 1,2,3,4,6-octa-O-galloyl-α-D-glucose, 1,2,3,4,6-octa-O-galloyl-β-D-glucose, 1,2,3,4,6-nona-O-galloyl-α-D-glucose, 1,2,3,4,6-nona-O-galloyl-β-D-glucose, 1,2,3,4,6-deca-O-galloyl-α-D-glucose, and 1,2,3,4,6-deca-O-galloyl-β-D-glucose.

In another embodiment, the method comprises contacting the preadipocytes with a biologically effective amount of a gallotannin variant composition comprising one or more gallotannin variants. In a further embodiment, the method comprises contacting the preadipocytes with both a hydrolysable gallotannin composition of the present invention and a gallotannin variant composition to the patient.

Procedure for Determining Dosage In Vitro

To determine the effective concentration of the gallotannin or gallotannin variant to use in preventing adipogenesis in vitro, undifferentiated preadipocytes are incubated either with a differentiation-induction cocktail, comprised of 3-isobutyl-1-methylxanthine, dexamethasone, and insulin (MDI); or with MDI plus the gallotannin or gallotannin variant. After about 10 days MDI induces differentiation, which is clearly visible as the change from fibroblast-like preadipcytes to round-shaped, fat vesicle-containing adipocytes. The degree of the differentiation of the cells is evaluated by microscopic observation of lipid accumulation and Oil Red 0 staining (only triglyceride containing vesicles can be stained red), as well as by the glucose uptake activities the treated cells exhibit at the end of the incubation period. The glucose uptake assay is chosen and performed here for determination of the degree of adipocyte differentiation based on the observation that differentiated adipocytes can be induced by insulin to take up glucose whereas preadipocytes cannot.

Procedure for Determining Dosage In Vivo

To determine the in vivo anti-adipogenic effect and effective dosage of the present gallotannin composition or variant gallotannin composition, genetically diabetic female mice (Type II, KK-A^{y}) of five weeks of age are used. The gallotannin or gallotanin variant is either orally delivered or IP injected daily into the mice at various concentrations for 6 to 10 weeks. The food intake and body weight of the mice are monitored. At the end of the experiment, the parametrial adipose tissues from the treated and control mice are removed, weighed, and compared. In addition, livers of the treated and the control mice are also removed, and the lipid contents of the livers are measured. The dosage that results in largest reduction in parametrial adipose tissue and hepatic lipid contents without significantly altering food intake is considered as optimal dosage for anti-adipogenic activity of the present gallotannin or gallotannin variant composition.

Exemplary Methods Of Making Hydrolysable Gallotannins

A. Isolation Methods

The beta forms of the hydrolysable gallotannins can be isolated from commercial tannic acid preparations using HPLC. HPLC system is Beckman System Gold consisting of a 125 solvent module, a 168 PDA detector and a 508 autosampler. For the separation, a Beckman Ultrasphere C-18 reversed phase Semi-Prep column (10.0 mm×250 mm I.D., 5 μm) is used. The detection wavelength is set at 320 nm, or 330 nm. Eluent A is water added 0.1% trifluoroacetic acid, eluent B is acetonitrile with 0.1% trifluoroacetic acid. A Foxy Jr. fraction collector from ISCO has been used to collect individual peaks in timed windows. The separation is achieved with isocratic gradients A:B 82:18 in 40 minutes at a flow rate of 3 mL/min. Under these conditions, the approximate retention time for the β forms of the gallotannins is as follows:

| | |
|---|---|
| Penta-O-galloyl-β-D-glucose | 13.5 minutes. |
| Hexa-O-galloyl-β-D-glucose | 20.2 minutes. |
| Octa-O-galloyl-β-D-glucose | 22.0 minutes. |
| Nona-O-galloyl-β-D-glucose | 30.0 minutes. |
| Deca-O-galloyl-β-D-glucose | 36.3 minutes. |
| Undeca-O-galloyl-β-D-glucose | 39.8 minutes. |
| B. Synthetic Methods | |

The method for the chemical synthesis of the alpha and beta forms of PGG consists of four steps:
(i) Starting with the methyl ester of gallic acid, the reaction with benzyl chloride in the presence of potassium carbonate and potassium iodide in acetone yields the protected gallic acid derivative methyl 3,4,5-tri-O-benzylgallate.
(ii) Ester cleavage with sodium hydroxide in aqueous ethanol leads to the formation of 3,4,5-tri-O-benzylgallic acid.
(iii) 3,4,5-Tri-O-benzylgallic acid is used in a dicyclohexylcarbodiimide-mediated esterification of D-glucose in the presence of 4-(dimethylamino)pyridine in 1,2-dichloroethane. Penta-O-(3,4,5-tri-O-benzylgalloyl)-D-glucopyranose is obtained as a mixture of α- and β-anomers.
(iv) Penta-O-(3,4,5-tri-O-benzylgalloyl)-D-glucopyranose is deprotected by hydrogenation in the presence of a palladium catalyst in tetrahydrofuran. Chromatotron separation of the a/P mix of PGG yields the individual clean anomers.

Identities and purities of natural abundance intermediates and products are determined by ¹H and ¹³C{¹H} NMR spectroscopy, electrospray mass spectrometry, and UV-visible spectra.

α-hexa-O-galloyl glucose, β-hexa-O-galloyl glucose, α-hepta-O-galloyl glucose, β-hepta-O-galloyl glucose, α-octa-O-galloyl glucose, β-octa-O-galloyl glucose, α-nona-O-galloyl glucose, β-nona-O-galloyl glucose, α-deca-O-galloyl glucose, β-deca-O-galloyl glucose can be made using a mixture of α-penta-O-galloyl glucose and β-penta-O-galloyl glucose as starting material. The addition reaction of one or more gallic acid to α/β-penta-O-galloyl glucose is the same or very similar to the reaction steps for synthesis of α/β-penta-O-galloyl glucose as described above. This reaction will synthesize a mixture of galloyl glucose with 6, 7, 8, 9, or 10 gallic acids. These different compounds can be separated into single species by HPLC, and their individual structural identity confirmed by mass spectra and NMR analyses.

α and β-tetra-O-galloyl glucose can be made by protecting one of the hydroxyl group on the glucose before addition of gallic acid, and deprotecting the hydroxyl after the addition reaction.

Exemplary Methods of Making Gallotannin Variants

### 1) Synthesis of Pentakis-O-(3,4-dihydroxybenzoyl)-β-D-glucopyranose-

### Step 1: Ethyl 3,4-dibenzyloxybenzoate

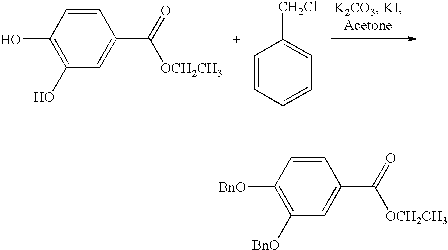 Procedure:

A mixture of ethyl 3,4-dihydroxybenzoate (10 g, 54.3 mmol), potassium iodide (4 g, 24 mmol), and anhydrous powdered potassium carbonate (40 g, 289 mmol) in acetone (500 mL) was stirred at room temperature for 20 min. Benzyl chloride (14.85 g, 117 mmol) dissolved in 100 mL acetone was added. The suspension was refluxed for 18 h. The solid was filtered off and the filtrate was evaporated. The residue was redissolved in 300 mL dichloromethane and filtered again. The solvent was evaporated. A dark yellow oil was obtained. It was used for the next step without any further purification.

### Step 2: 3,4-dibenzyloxybenzoic acid

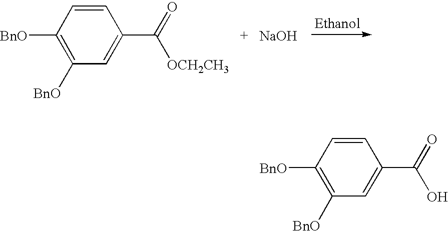 Procedure:

The crude product of the previous step was suspended in 95% ethanol (300 mL) and sodium hydroxide pellets (3.54 g, 88.5 mmol) were added. The mixture was heated under reflux for 3 h. The hot solution was poured into a mixture of 500 mL water and 25 mL concentrated hydrochloric acid. After swirling the flask for 10 min, the product was filtered off and successively washed with water (100 mL), 95% ethanol (100 mL), methanol (100 mL), and methl tert-butyl ether (100 mL). The white solid was dried overnight at room temperature in an oil pump vacuum (−0.1 bar).

Yield over two steps: 18.6 g (93%)

### Step 3: Pentakis-O-(3,4-dibenzyloxybenzoyl)-β-D-glucopyranose

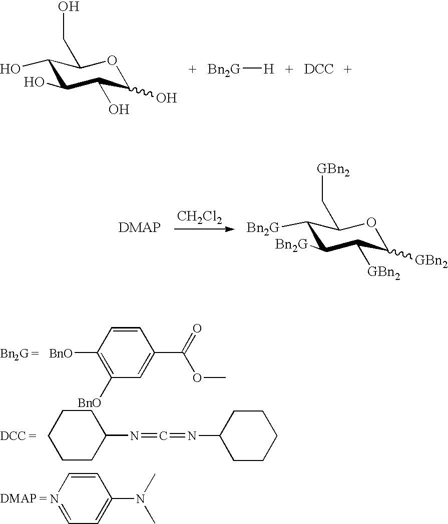 Procedure:

D-Glucose (0.2 g, 1.11 mmol), 3,4-dibenzyloxybenzoic acid (2.78 g. 8.31 mmol), dicyclohexylcarbodiimide (DCC, 1.84 g, 8.92 mmol), and N,N-Dimethylaminopyridine (DMAP, 1.08 g, 8.84 mmol) were added to dry dichloromethane (130 mL). The suspension was refluxed for 2.5 days. After cooling to room temperature, the urea byproduct was filtered off. 8 g of silica gel were added to the filtrate and it was evaporated to dryness. The residue was applied to a silica gel column (solvent system: dichloromethane:toluene:ethyl acetate 10=300:100:4). Clean fractions were combined and evaporated.

Yield: 1.72 g (88%) highly viscous clear oil.

### Step 4: Pentakis-O-(3,4-dihydroxybenzoyl)-β-D-glucopyranose

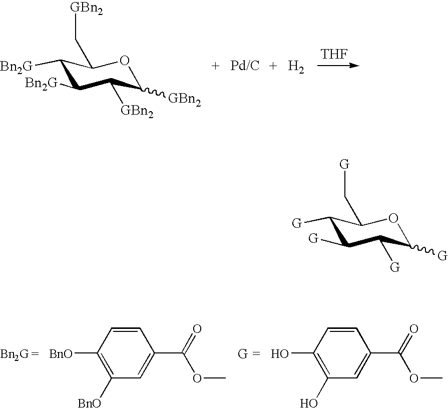 Procedure:

The benzyl protected starting material (392 mg, 0.222 mmol) was dissolved in dry THF (50 mL). The solution was degassed by applying a water aspirator vacuum for about 30 seconds while stirring magnetically. The flask was then flushed with argon gas. Degasing and flushing were repeated two more times. 10% Palladium on charcoal (287 mg, 0.27 mmol) was added. The mixture was degassed and then flushed with hydrogen gas. The degassing and flushing was repeated two more times. The suspension was then stirred at maximum speed at 40° C. under a hydrogen gas atmosphere at normal pressure for 5 h. The mixture was cooled, filtered through Celite, and the filtrate was evaporated.

Yield: 190 mg (99%) of a foamy amorphous solid.

Other gallotannin variants that comprise a dihydroxybenzyloxybenzoyl moiety are made as described above except that a different ethyl dihydroxybenzoate is used as the starting material of step 1.

### 2) Synthesis of Tetrakis-O-(3,4-dihydroxybenzoyl)-β-D-glucopyranose

### Step 1: Methyl 3,4,5-tribenzyloxybenzoate

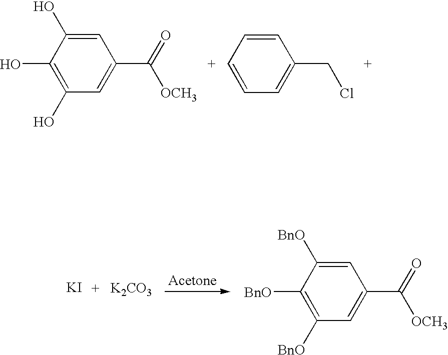 Procedure:

A mixture of methyl 3,4,5-trihydroxybenzoate (10 g, 54.3 mmol), potassium iodide (4 g, 24 mmol), and anhydrous powdered potassium carbonate (44 g, 318 mmol) in acetone (500 mL) was stirred at room temperature for 20 min. Benzyl chloride (22 g, 174 mmol) dissolved in 100 mL acetone was added. The suspension was refluxed for 18 h. The solid was filtered off and the filtrate was evaporated The residue was taken up in 400 mL dichloromethane. The suspension was filtered through celite and the filtrate was evaporated. The residue was dried at room temperature in an oil pump vacuum for 45 min.

Yield: 26.528 g (107%) (the product contains some benzyl chloride as an impurity)

### Step 2: 3,4,5-Tribenzyloxybenzoic acid

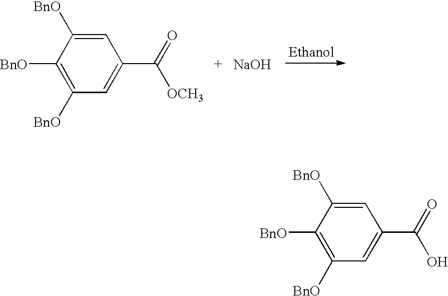 Procedure:

The crude product of the previous step was suspended in 95% ethanol (300 mL) and sodium hydroxide pellets (3.54 g, 88.5 mmol) were added. The mixture was heated under reflux for 3 h. The hot solution was poured into a mixture of 500 mL water and 25 mL concentrated hydrochloric acid. After swirling the flask for 10 min, the product was filtered off and successively washed with water (100 mL), 95% ethanol (100 mL), methanol (100 mL), and methl tert-butyl ether (100 mL). The white solid was dried overnight at room temperature in an oil pump vacuum (˜0.1 bar).

Yield over two steps: 22.42 g (94%)

### Step 3: Tetrakis-O-(3,4,5-tribenzyloxybenzoyl)-D-xylopyranose

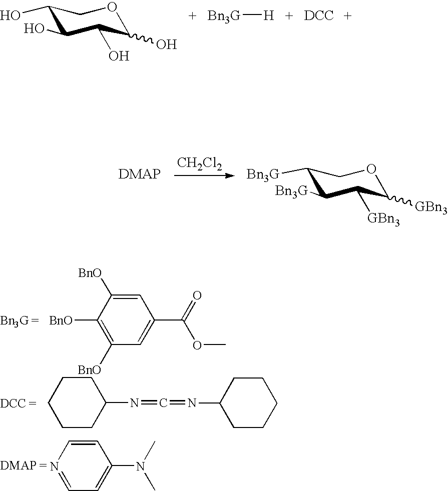 Procedure:

D-Xylose (0.2 g, 1.33 mmol), 3,4,5-tribenzyloxybenzoic acid (3.515 g. 7.98 mmol), dicyclohexylcarbodiimide (DCC, 1.77 g, 8.57 mmol), and N,N-Dimethylaminopyridine (DMAP, 1.04 g, 8.49 mmol) were added to dry dichloromethane (130 mL). The suspension was refluxed for 2.5 days. After cooling to room temperature, the urea byproduct was filtered off. 9 g of silica gel were added to the filtrate and it was evaporated to dryness. The residue was applied to a silica gel column (solvent system: dichloromethane:toluene:ethyl acetate=300:100:4). Clean fractions were combined and evaporated.

Yield: 208 mg of the beta isomer
220 mg of the alpha isomer
770 mg of mixed fractions (alpha+beta)

### Step 4: Tetrakis-O-(3,4,5-trihydroxybenzoyl)-β-D-glucopyranose

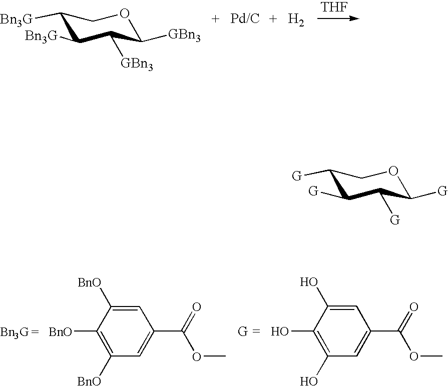 Procedure:

The benzyl protected starting material (150 mg, 0.0815 mmol) was dissolved in dry THF (20 mL). The solution was degassed by applying a water aspirator vacuum for about 30 seconds while stirring magnetically. The flask was then flushed with argon gas. Degasing and flushing were repeated two more times. 10% Palladium on charcoal (120 mg, 0.113 mmol) was added. The mixture was degassed and then flushed with hydrogen gas. The degassing and flushing was repeated two more times. The suspension was then stirred at maximum speed at 40° C. under a hydrogen gas atmosphere at normal pressure for 5 h. The mixture was cooled, filtered through Celite, and the filtrate was evaporated.

Yield: 62 mg (100%) of a foamy amorphous solid.

Other gallotannin variants comprising a sugar core other than glucose, e.g. a galactose, mannose, trehalose, maltose, cellobiose, inositol, and glucitol, are made as described above except that the xylose that is added in step 3 is replaced with another sugar.

3) Replacing the Ester Linkage Between Gallic Acid and Glucose with an Ether Linkage:

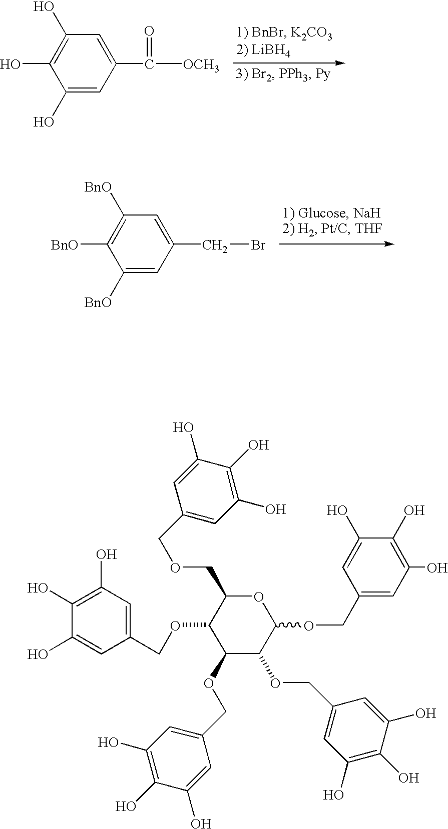

The first three steps of the synthesis are literature procedures (E. Eich, H. Pertz, M. Kaloga, J. Schulz, M. R. Fesen, A. Mazumder, Y. Pommier, (−)-Arctigenin as a Lead Structure for Inhibitors of Human Immunodeficiency Virus Type-1 Integrase, J. Med. Chem. 1996, 39, 86-95).

The subsequent steps are analogous to standard benzyl protection/deprotection chemistry of carbohydrates. The final hydrogenolysis will be much faster for the phenolic benzyl groups than for the carbohydrate bound trihydroxybenzyl groups. It is expected that the decreased sensitivity of the ether linkage to acid hydrolysis will increase the stability (half life) of the molecule, and hence the duration of action and overall apparent biological activity in vivo.

### EXAMPLES

The following examples are for purposes of illustration only and are not intended to limit the scope of the claims which are appended hereto. All references cited herein are specifically incorporated in their entirety herein.

### Example 1

### Stimulation of Glucose Uptake in Cells by Penta-O-Galloyl-D-Glucose (PGG)

A 50:50 mixture of α-PGG and β-PGG was synthesized as described above. The alpha and beta anomers were separated as described above. The glucose transport stimulatory activity of the two anomoers was compared to that of authentic plant derived β-PGG. Chemically synthesized PGG and authentic plant derived PGG are spectrally identical.

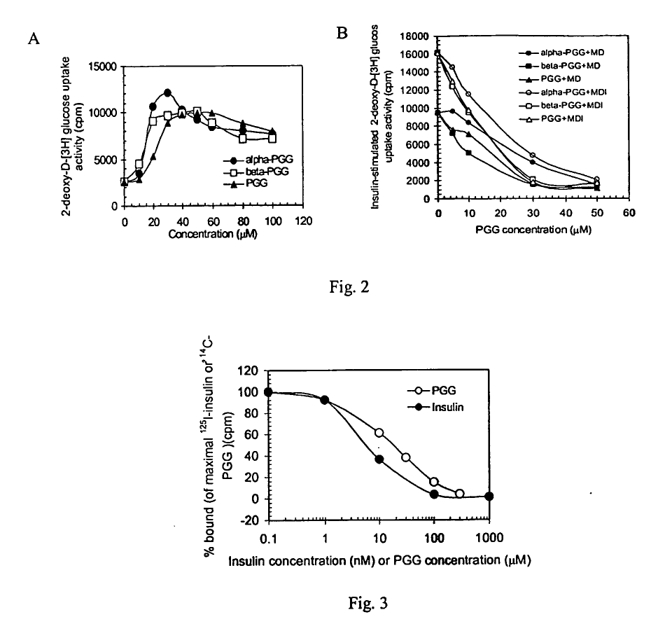

3T3-L1 adipocytes were purchased from ATCC, and maintained and passed as preadipocytes in DMEM supplemented with 10% calf serum in a 37° C. incubator with 10% CO₂ as required by the cells. The cells were induced to differentiate into adipocytes by addition of MDI induction cocktail as described in Liu, F., Kim, J., Li, Y., Liu, X., Li, J. & Chen, X. (2001) An extract of Lagerstroemia speciosa L. has insulin-like glucose uptake-stimulatory and adipocyte differentiation-inhibitory activities in 3T3-L1 cells. J. Nutrition 131:2242-224, which is specifically incorporated herein by reference. Varying amounts of α-PGG and β-PGG were then added to the medium and the amount of glucose taken up by control and treated cells determined using a standard glucose uptake assay, as described in Liu et al. As shown in FIG. 2A chemically synthesized and plant derived β-PGG have similar glucose transport stimulatory (GTS). The GTS activity of α-PGG is consistently 10-20% higher than that of β-PGG (FIG. 2).

To investigate the mechanism by which PGG induces GTS activity, ¹⁴C-labeled PGG was synthesized and used in cell studies. To determine if PGG acts at the cell membrane or intracellularly, radioactive PGG was incubated with 3T3-L1 adipocytes at either 37° C. or 4° C. for various times. Following incubation, the excess radioactive PGG was removed by washings and the cells were harvested and were separated into cell membrane and intracellular fractions using published protocols. More than 90-95% of the radioactivity was associated with the membrane fraction, suggesting PGG interacts with 3T3-L1 cells by binding to the cell membrane.

Ligand displacement studies were carried out using 3T3-L1 cells incubated overnight at 4° C. with ¹⁴C-PGG and increasing amounts of unlabeled PGG. Radioactive insulin was used as a receptor binding control. In the concentration range tested, PGG appeared to function as a ligand, binding to a target with an apparent receptor binding constant (Kd) of ˜10⁻⁵ M (FIG. 3). It is worth noting that the estimated Kd is very similar to the EC50s of PGG for both GTS and AD activities (FIG. 2). Since PGG possesses an insulin-like GTS activity and appears to induce the GTS activity in a way that is similar to insulin, it was speculated that PGG induces the GTS activity in 3T3-L1 cells by binding to the insulin receptor.

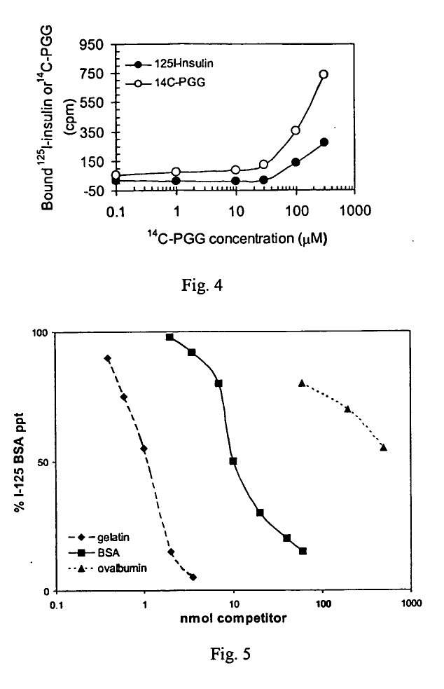

To determine whether PGG and insulin have a common target, 3T3-L1 cells were incubated with a constant concentration of radioactive insulin plus increasing amounts of cold PGG, or with radioactive PGG with increasing amounts of cold insulin. The binding assay results indicate that PGG does not compete with insulin for the insulin binding site located on IR (FIG. 4). Conversely, insulin was also found not to compete with PGG for the PGG binding site located on the cell membrane. Thus, PGG seems to bind either to a site on IR other than the insulin binding site, or to a membrane receptor other than IR.

A competitive binding assay using radiolabeled bovine serum albumin (BSA) as the tracer was used to determine whether PGG can bind proteins selectively. The relative affinities of three standard proteins for PGG differ from one another by at least 10-fold (FIG. 5), and the difference of the binding affinities of PGG to gelatin and ovalbumin is more than 100-fold. This indicates that PGG-protein interactions have specificity and that PGG could selectively act at a single biochemical target such as IR.

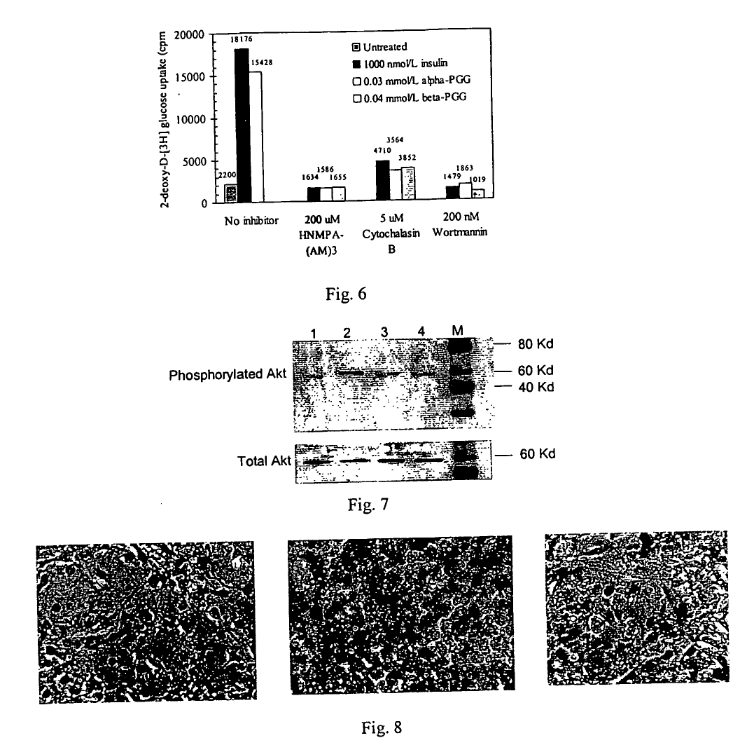

Chemical inhibitors which specifically inhibit proteins/enzymes involved in the insulin-mediated signaling pathway, were used to further identify the molecular target of PGG. Three inhibitors were tested: hydroxyl-2-naphthalenylmethylphosphonic acid tri-acetoxymethyl ester (HNMPA-(AM)₃, a chemical that inhibits IR tyrosine kinase (Qiu, Z., et al. (2001) J. Biol. Chem. 276:11988-11995); wortmannin, a compound that specifically inhibits PI-3K (Saperstein, R., et al. (1989) Biochemistry 28:5694-5701); and cytochalasin B, a compound that specifically inhibits glucose transport mediated by GLUT4 (Tomiyama, K., et al. (1995) Biochem. Biophy. Res. Commu. 212:263-269; Kletzien, R. F. et a. (1972) J. Biol. Chem. 247:2964-2966). All three inhibitors completely inhibited the GTS activity mediated by either insulin or PGG (FIG. 6), suggesting that all the GTS activity mediated by PGG is through, and only through the insulin-mediated signaling pathway. The fact that HNMPA-(AM)₃, which is an inhibitor of the first enzyme in the insulin signaling pathway (IR tyrosine kinase,), also completely abolished the GTS activity of PGG, suggests that the molecular target of PGG is IR. The notion that PGG uses insulin-mediated GTS pathway was further supported by our Western blot analysis that shows Akt, a key protein kinase involved in the pathway (zz), is phosphorylated by PGG (FIG. 7).

### Example 2

### Effect of PGG on Adipogenesis

3T3-L1 adipocytes were purchased from ATCC, and maintained and passed as preadipocytes in DMEM supplemented with 10% calf serum in a 37° C. incubator with 10% CO₂ as required by the cells. To test the effect of PGG on adipogenesis, the preadipocytes were incubated either with a differentiation-induction cocktail comprised of 3-isobutyl-1-methylxanthine, dexamethasone(MDI), a cocktail comprised of 3-isobutyl-1-methylxanthine, dexamethasone (MD) and PGG; or with MDI plus PGG. When insulin in MDI was substituted by PGG, the new cocktail failed to induce differentiation of the preadipocytes. (See FIG. 2 B.) These results indicate that PGG cannot replace insulin for induction differentiation of pre-adipocytes to adipocytes.

A cell proliferation assay was used to determine if PGG inhibits adipocyte differentiation by blocking clonal expansion. The assay indicated that the first round of clonal expansion is not inhibited by either α- or β-PGG. The second round of clonal expansion is partially inhibited by α-PGG, and completely inhibited by β-PGG (FIG. 10). The basis for the difference between the anomers in clonal expansion and differentiation is not known. However, our results indicate that clonal expansion inhibition or apoptosis (programmed cell death) in preadipocytes does not account for the differentiation inhibition observed, since (X-PGG abolishes the differentiation with only limited inhibition of the clonal expansion and without significantly increased cell-killing compared to MDI induced cells (data not shown). Thus, the key step of the inhibition is likely to be at a step that is different from the clonal expansion.

In order to study the mechanism by which PGG inhibits 3T3-L1 preadipocyte differentiation, we used gene expression studies. This strategy allows us to determine whether PGG specifically acts on genes that participate in the differentiation process. In addition, the gene expression study overcomes difficulties of studying the insulin receptor in preadipocytes, which express 10 times few IR on their cell surface (Modan-Moses, et al. (1998) Biochem. J. 333:825-831). To investigate genes affected by PGG during AD, preadipocytes were incubated either with a differentiation-induction cocktail, comprised of 3-isobutyl-1-methylxanthine, dexamethasone, and insulin; or with MDI plus PGG. After about 10 days MDI induces differentiation, which is clearly visible as the change from fibroblast-like preadipcytes to round-shaped, fat vesicle-containing adipocytes (FIG. 8 middle). In contrast, preadipocytes treated with MDI plus PGG retain their fibroblast-like morphology and remain fat vesicle-free (FIG. 8 right).

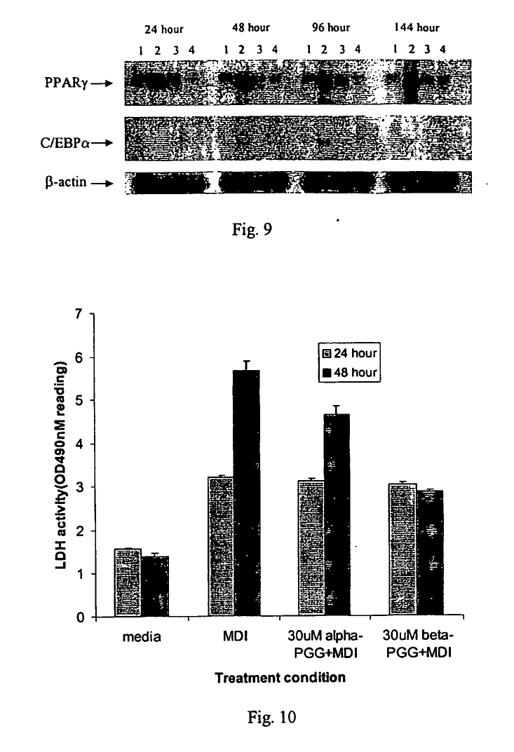

At various times the treated cells were harvested, the total RNA was isolated, and expression was analyzed by Northern blots using probes complementary to various genes that are involved in the differentiation process. Northern blot analyses revealed that the expression of genes PPAR-γ, c/EBP-t, which are required for the differentiation and are induced by MDI, were completely abolished by PGG (FIG. 9). Relatively consistent β-actin level in differentially treated cells indicates that other cell processes such as β-actin expression is not significantly affected by PGG (FIG. 9).

### Example 3

### Effect on PGG on Reducing Blood Glucose Levels in Diabetic Animals

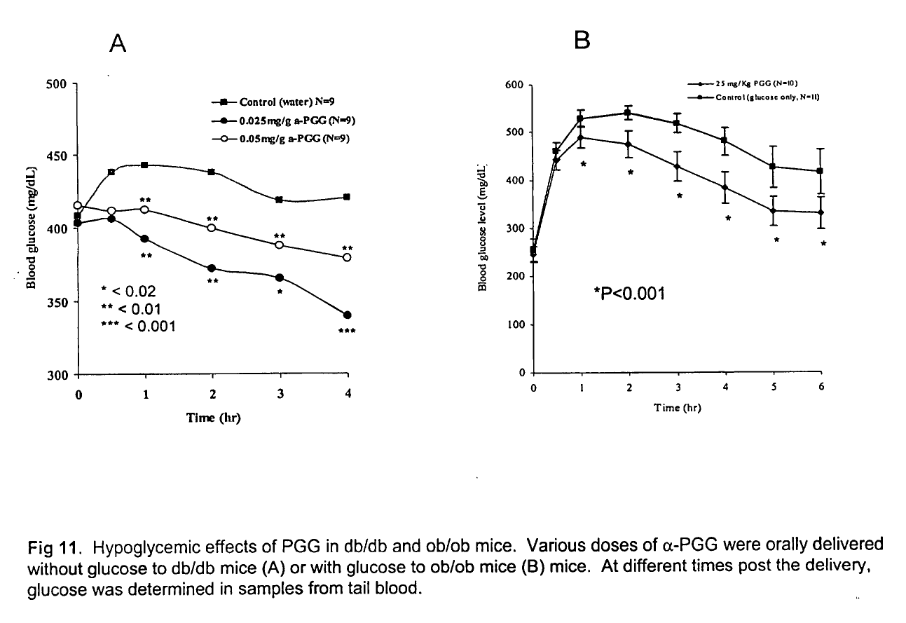

To determine whether α-PGG could exhibit anti-diabetic activities in vivo, α-PGG in the form of an aqueous solution was orally delivered to 8-week old male fasting diabetic db/db mice. It was found that a single dose of α-PGG at a concentration of 25 mg/kg body weight significantly reduced the blood glucose levels in db/db mice compared to the db/db mice received vehicle (same aqueous solution without α-PGG) (FIG. 11A). The reduction of the glucose level is about 15-20% depending on the time post α-pGG administration (P<0.01, FIG. 11A).

To determine if α-PGG is also effective in improving glucose tolerance in diabetic and obese mice, a glucose tolerance test was performed using ob/ob mice. Glucose or glucose plus α-PGG were orally delivered into male ob/ob mice, and blood glucose levels were measured at various times post glucose/PGG administration. The ob/ob mice receiving glucose plus α-PGG have significantly lower blood glucose levels compared to those mice treated with glucose alone (P<0.001, FIG. 11B). Interestingly, the improved blood glucose levels could still be observed 24 hrs after the α-PGG treatment, indicating the effect of α-PGG is relatively long-lasting.

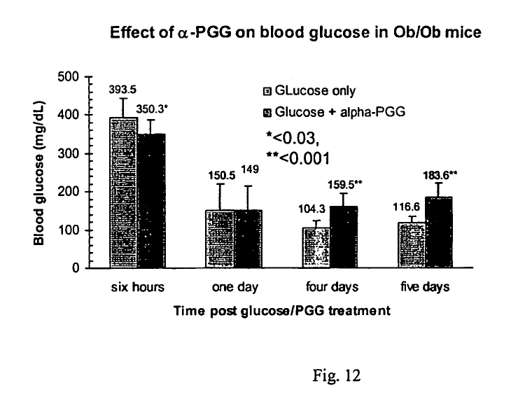

A single high does of glucose in ob/ob mice can be fatal, either because of the very high levels of blood glucose levels ˜24 hrs after the glucose challenge or because of the abnormally low glucose levels 2-3 days after the glucose challenge. We found that α-PGG not only protected ob/ob mice from extremely high glucose levels right after the glucose challenge (FIG. 11B) but also protected these mice from suffering extremely low glucose levels 2-5 days later (FIG. 12). Although the protection mechanisms are unknown at this time, it is very likely that α-PGG protected the ob/ob mice not only by its GTS activity, but also some other activity such as AD-related activity. No PGG-related toxic effects were observed in these mice.

These animal studies clearly demonstrate that α-PGG not only works in 3T3-L1 cells, but that it is also effective in vivo in diabetic/obese animal models.

### Example 4

### Effect of Gallotannin Variants on Glucose Uptake by Cells

We have tested several classes of polymeric polyphenolics for GTS in the 3T3-LT1 cell system (Table 1). Banaba extract and the gallotannin preparations (tannic acid, a mixture of galloyl glucoses; and purified PGG) had high activities. The proanthocyanidins had limited activity and the tea catechin EGCG had no activity. This supports the idea that specific protein-PGG interactions are responsible for the GTS activity, since only PGG and closely related compounds were active. If GTS activity were simply a result of general protein binding by polymeric polyphenolics, then the flavonoid-based proanthocyanidin and tea catechin preparations would be active.

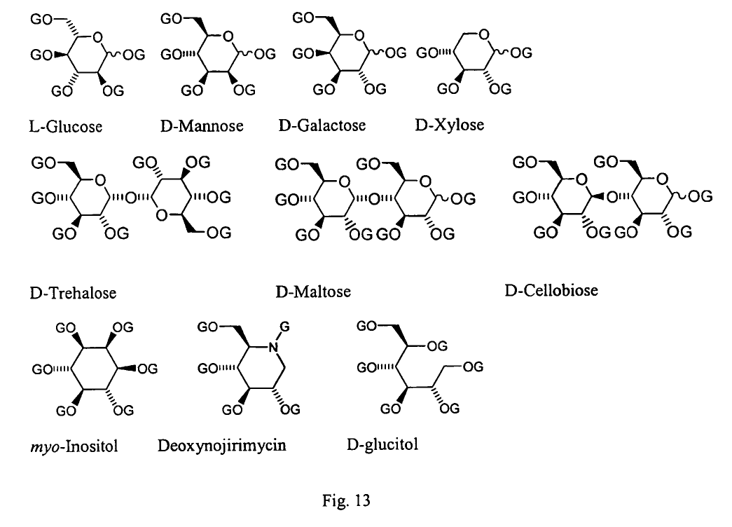

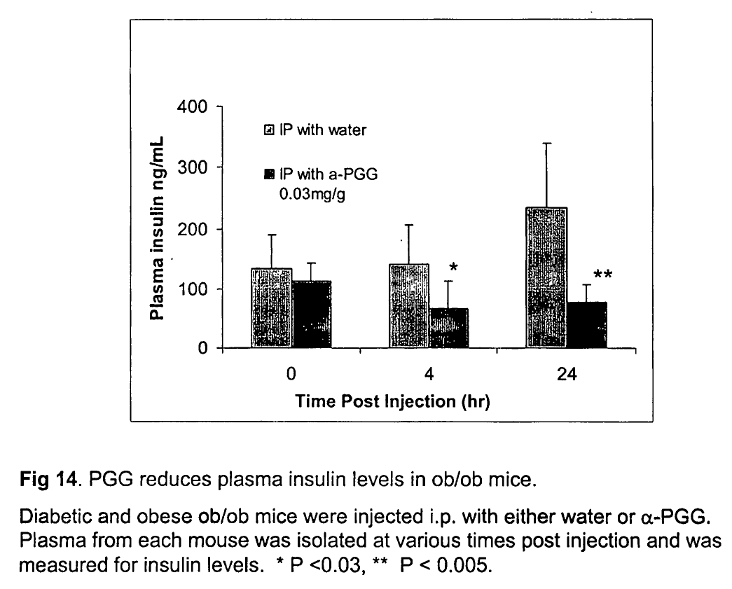

Reproducible difference in GTS and AD activities between α-PGG and β-PGG (FIG. 2) and between PGG and hexa-GG (Table 1) were routinely obtained. It seems likely that the orientation of the gallic acid at carbon 1 of the sugar is responsible for the difference in activity observed between α-PGG and β-PGG. Pentagalloyl galactose (PGGal), a gallotannin variant of the present invention, and pentagalloyl deoxynojirimycin (PG-DJM) (FIG. 13) were synthesized. We found that PGGal is 60-70% as active as PGG whereas PG-DJM has no detectable GTS activity.

TABLE
Glucose Transport Stimulatory Activity of PGG Derivatives

| PGG derivative | Structure | G component | GTS activity |
|---|---|---|---|
| 1) β-Glc(G-3,5-OH2)5 Pentakis-O-(3,5-dihydroxybenzoyl) - β-D-glucopyranose | 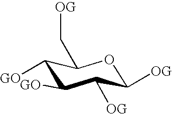 | 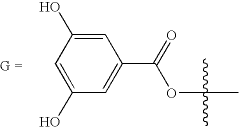 | − |
| 2) β-Glc(G-3,4-OH2)5 Pentakis-O-(3,4-dihydroxybenzoyl) -β-D-glucopyranose | 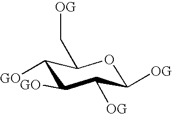 | 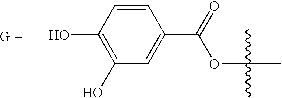 | ++* |
| 3) α-Glc(G-3,5-OH2)5 Pentakis-O-(3,5-dihydroxybenzoyl) -α-D-glucopyranose | 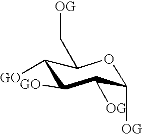 | 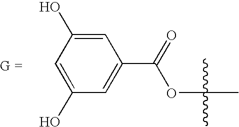 | − |
| 4) β-XylG4 Tetrakis-O-(3,4,5-trihydroxybenzoyl) -β-D-xylopyranose | 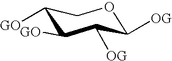 | 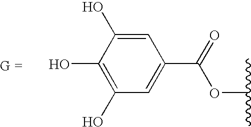 | − |
| 5) α-XylG4 Tetrakis-O-(3,4,5-trihydroxybenzoyl) -α-D-xylopyranose | 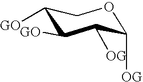 | 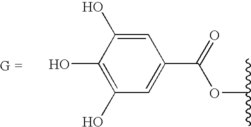 | +++ |

*α-PGG's GTS activity is +++++, and β-PGG's activity is ++++.

### Example 5

### Effect of PGG on Plasma Insulin Levels

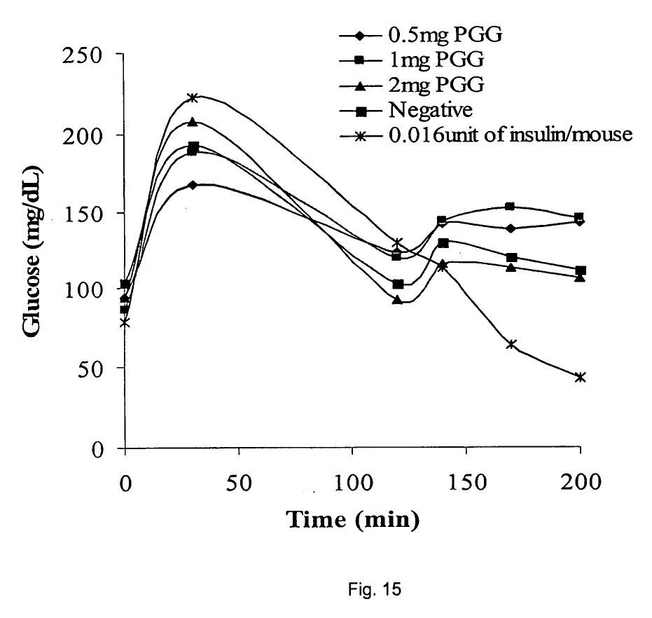

Diabetic and obese ob/ob mice were injected intraperitoneally with either water or α-PGG. Plasma from each mouse was isolated at various times post injection and was measured for insulin levels. As shown in FIG. 15, ob/ob diabetic and obese mice treated with a single injection of α-PGG had significantly lower plasma insulin levels than ob/ob diabetic mice treated with water alone (negative controls). On the basis of these results and PGG's effect on glucose uptake, it is believed that, in vivo, PGG can enhance the glucose transport stimulatory activity of insulin. Thus, it is expected that PGG can be used therapeutically to improve insulin resistance in a mammalian subject

### Example 6

### Effect of Pentakis-O-(3,4 dihydroxybenzoyl)-B-D-glucopyranose and Tetrakis-O-(3,4,5, trihydoxybenzoyl-α-D)xylopyranose on glucose uptake by adipocytes

Confluent 3T3-L1 adipocytes grown in 12-well plates were washed twice with serum-free DMEM and incubated with 1 mL of the same medium at 37° C. for 2 h. The cells were washed 3 times with Krebs-Ringer-Hepes (KRP) buffer and incubated with 0.9 mL KRP buffer at 37° C. for 30 min. Compounds listed in the table below were then added at 20-40 μM (final concentration?) and adipocytes were incubated at 37° C. for 15 min. Glucose uptake was initiated by addition of 0.1 mL KRP buffer and 37 MBq/L 2-deoxy-D-[3H] glucose and 1 mmol/L glucose as final concentrations. After 10 min, glucose uptake was terminated by washing the cells 3 times with cold PBS. The cells were lysed with 0.7 mL of 1% Triton X-100 at 37° C. for 20 min. The radioactivity retained by the cell lysates is determined by a scintillation counter. As shown in the table the gallotannin variants Pentakis-O-(3,4 dihydroxybenzoyl)-β-D-glucopyranose and Tetrakis-O-(3,4,5, trihydoxybenzoyl)-α-D-xylopyranose increased glucose uptake by the cells

### Example 7

### PGG Does Not Cause Hypoglycemia

Normal (healthy) mice were orally fed with 40 mg of glucose at time zero. After two hours (120 min) when blood glucose were at normal or basal levels, mice were orally fed with either PGG at various concentrations, or water (negative control), or were injected intraperitonealy with insulin. At various time intervals before and after administration of PGG or insulin, blood was collected from the control and treated animals and blood glucose levels determined. As shown in the FIG. 15, insulin injection resulted in a hypoglycemic condition in mice, whereas PGG administration did not. Thus, PGG reduces blood glucose level when it is higher than normal. However, PGG does not further reduce bood glucose levels beyond normal or basal levels.

What is claimed is:

1. A method for treating or preventing diabetes in a mammalian subject, comprising:
administering a therapeutically effective amount of a gallotannin composition to a subject in need of the same,
wherein said gallotannin composition comprises one or more hydrolysable gallotannins selected from the group consisting of 1,2,3,4-tetra-O-galloyl-α-D-glucose, 1,2,3,6-tetra-O-galloyl-α-D-glucose, 1,3,4,6-tetra-O-galloyl-α-D-glucose, 1,2,3,4,6-penta-O-galloyl-α-D-glucose, 1,2,3,4,6-penta-O-galloyl-β-D-glucose, 1,2,3,4,6-hexa-galloyl-α-D-glucose, 1,2,3,4,6-hexa-O-galloyl-β-D-glucose, 1,2,3,4,6-hepta-O-galloyl-α-D-glucose, 1,2,3,4,6-hepta-O-galloyl-β-D-glucose, 1,2,3,4,6-octa-O-galloyl-α-D-glucose, 1,2,3,4,6-octa-O-galloyl-β-D-glucose, 1,2,3,4,6-nona-O-galloyl-β-D-glucose, 1,2,3,4,6-nona-O-galloyl-β-D-glucose, 1,2,3,4,6-deca-O-galloyl-α-D-glucose, and 1,2,3,4,6-deca-O-galloyl-β-D-glucose, or a salt thereof, and
wherein said gallotannin composition comprises less than 5% by dry weight of one or more of the following compounds: mono-O-galloyl-β-D-glucose, di-O-galloyl-β-D-glucose, tri-O-galloyl-β-D-glucose, tetra-O-galloyl-β-D-glucose, unadeca-O-galloyl-β-D-glucose, dodeca-O-galloyl-β-D-glucose or mixtures thereof.

2. The method of claim 1 wherein said gallotannin composition comprises at least 50% by dry weight of 1,2,3,4,6-penta-O-galloyl-α-D-glucose or 1,2,3,4,6-penta-O-galloyl-β-D-glucose or a combination of 1,2,3,4,6-penta-O-galloyl-α-D-glucose and 1,2,3,4,6-penta-O-galloyl-β-D-glucose.

3. The method of claim 1 wherein said gallotannin composition comprises at least 50% by dry weight of 1,2,3,4,6-penta-O-galloyl-α-D-glucose.

4. The method of claim 1 wherein said gallotannin composition comprises at least 50% by dry weight of 1,2,3,4,6-penta-O-galloyl-β-D-glucose.

5. The method of claim 1 wherein said gallotannin composition is administered to the subject orally or by injection.

6. The method of claim 1 further comprising the step of administering insulin to the subject.

7. A method for treating or preventing diabetes in a subject, comprising:
administering a gallotannin variant composition to the subject,
wherein said gallotannin variant composition comprises one or more gallotannin variant compounds or a salt thereof, wherein each of said gallotannin variant compounds has the following structure:
R—X-A(ₙ)-X-A(_{q})-X-A(_{z}),
wherein R is selected from the group consisting of D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose, α-D-Xylose, α-D Lyxose, β-D Lyxose, α-D Arabinose, β-D Arabinose, α-D Ribose, β-D Ribose, D-Trehalose, D-Maltose, D-Cellobiose, myo-Inositol, D-glucitol,
X is an ester or ether linkage,
A is a trihydroxybenzoic acid selected from the group consisting of 3,4,5-trihydroxybenzoic acid, 2,3,4-trihydroxybenzoic acid 2,4,6-trihydroxybenzoic acid, or a dihydroxybenzoic acid selected from the group consisting of 2,3-dihydroxybenzoic acid, 2,4-dihydroxybenzoic acid, 3,4-dihydroxybenzoic acid, or a monohydroxybenzoic acids selected from the group consisting of 3-hydroxybenzoic acid and 4-hydroxybenzoic acid,
n is 5, q is 0, 1, 2, 3, 4, or 5, and z is 0 when R is D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose;
n is 4, q is 0, 1, 2, 3, or 4, and z is 0, 1, or 2 when R is α-D-Xylose, α-D Lyxose, E-D Lyxose, α-D Arabinose, E-D Arabinose, α-D Ribose, β-D Ribose;
n is 6, q is 0, 1, 2, 3, 4, 5, or 6, and z is 0 when R is D-Glucitol or myo-Inositol, and
n is 8, q is 0, 1, 2, 3, 4, 5, 6, 7, or 8, and z is 0 when R is D-Trehalose, D-Maltose, D- or Cellobiose.

8. The method of claim 7 wherein X is an ether linkage.

9. The method of claim 7 wherein R is L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose, α-D-Xylose, α-D Lyxose, β-D Lyxose, α-D Arabinose, β-D Arabinose, α-D Ribose, β-D Ribose, D-Trehalose, D-Maltose, D-Cellobiose, myo-Inositol, or D-glucitol.

10. The method of claim 4 wherein A is a trihydroxybenzoic acid selected from the group consisting of 2,3,4-trihydroxybenzoic acid 2,4,6-trihydroxybenzoic acid, or a dihydroxybenzoic acid selected from the group consisting of 2,3-dihydroxybenzoic acid, 2,4-dihydroxybenzoic acid, 3,4-dihydroxybenzoic acid, or a monohydroxybenzoic acids selected from the group consisting of 3-hydroxybenzoic acid and 4-hydroxybenzoic acid.

11. The method of claim 4 wherein each of said gallotannin variant compounds has a structure other than the structure tetra-O-galloyl-β-D-glucose, 1,2,3,4,6-penta-O-galloyl-β-D-glucose, 1,2,3,4,6-hexa-O-galloyl-β-D-glucose, 1,2,3,4,6-hepta-O-galloyl-β-D-glucose, 1,2,3,4,6-octa-O-galloyl-β-D-glucose, 1,2,3,4,6-nona-O-galloyl-β-D-glucose, and 1,2,3,4,6-deca-O-galloyl-β-D-glucose.

12. A method of reducing elevated blood glucose levels in a subject in need thereof without causing hypoglycemia in the subject, comprising:
administering one or both of the following compositions (a) and (b) to the subject,
(a) a gallotannin composition which comprises one or more hydrolysable gallotannins selected from the group consisting of wherein said gallotannin composition comprises one or more hydrolysable gallotannins selected from the group consisting of 1,2,3,4-tetra-O-galloyl-α-D-glucose, 1,2,3,6-tetra-O-galloyl-α-D-glucose, 1,3,4,6-tetra-O-galloyl-α-D-glucose, 1,2,3,4,6-penta-O-galloyl-α-D-glucose, 1,2,3,4,6-penta-O-galloyl-β-D-glucose, 1,2,3,4,6-hexa-galloyl-α-D-glucose, 1,2,3,4,6-hexa-O-galloyl-β-D-glucose, 1,2,3,4,6-hepta-O-galloyl-α-D-glucose, 1,2,3,4,6-hepta-O-galloyl-β-D-glucose, 1,2,3,4,6-octa-O-galloyl-α-D-glucose, 1,2,3,4,6-octa-O-galloyl-β-D-glucose, 1,2,3,4,6-nona-O-galloyl-α-D-glucose, 1,2,3,4,6-nona-O-galloyl-β-D-glucose, 1,2,3,4,6-deca-O-galloyl-α-D-glucose, and 1,2,3,4,6-deca-O-galloyl-β-D-glucose, or a pharmaceutically acceptable salt thereof, and
wherein said gallotannin composition comprises less than 5% by dry weight of one or more of the following compounds: mono-O-galloyl-β-D-glucose, di-O-galloyl-β-D-glucose, tri-O-galloyl-β-D-glucose, tetra-O-galloyl-β-D-glucose, unadeca-O-galloyl-β-D-glucose, dodeca-O-galloyl-β-D-glucose or mixtures thereof;
(b) a gallotannin variant composition which comprises one or more compounds having the following structure, or a pharmaceutically acceptable salt thereof:
R—X-A(ₙ)-X-A(_{q})-X-A(_{z}),
wherein R is selected from the group consisting of D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose, α-D-Xylose, α-D Lyxose, β-D Lyxose, α-D Arabinose, β-D Arabinose, α-D Ribose, β-D Ribose, D-Trehalose, D-Maltose, D-Cellobiose, myo-Inositol, D-glucitol,
X is an ester or ether linkage,
A is a trihydroxybenzoic acid selected from the group consisting of 3,4,5-trihydroxybenzoic acid, 2,3,4-trihydroxybenzoic acid 2,4,6-trihydroxybenzoic acid, or a dihydroxybenzoic acid selected from the group consisting of 2,3-dihydroxybenzoic acid, 2,4-dihydroxybenzoic acid, 3,4-dihydroxybenzoic acid, or a monohydroxybenzoic acids selected from the group consisting of 3-hydroxybenzoic acid and 4-hydroxybenzoic acid,
n is 5, q is 0, 1, 2, 3, 4, or 5, and z is 0 when R is D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose;
n is 4, q is 0, 1, 2, 3, or 4, and z is 0, 1, or 2 when R is α-D-Xylose, α-D Lyxose, β-D Lyxose, α-D Arabinose, β-D Arabinose, α-D Ribose, β-D Ribose;
n is 6, q is 0, 1, 2, 3, 4, 5, or 6, and z is 0 when R is D-Glucitol or myo-Inositol, and
n is 8, q is 0, 1, 2, 3, 4, 5, 6, 7, or 8, and z is 0 when R is D-Trehalose, D-Maltose, or D-Cellobiose.

13. A method of treating a subject exhibiting one or more of obesity, type II diabetes, glucose intolerance, insulin resistance, hyperglycemia, or hyperinsulinemia comprising
administering a gallotannin composition to the subject,
wherein said gallotannin composition comprises one or more hydrolysable gallotannins selected from the group consisting of wherein said gallotannin composition comprises one or more hydrolysable gallotannins selected from the group consisting of 1,2,3,4-tetra-O-galloyl-α-D-glucose, 1,2,3,6-tetra-O-galloyl-α-D-glucose, 1,3,4,6-tetra-O-galloyl-α-D-glucose, 1,2,3,4,6-penta-O-galloyl-α-D-glucose, 1,2,3,4,6-penta-O-galloyl-α-D-glucose, 1,2,3,4,6-hexa-galloyl-α-D-glucose, 1,2,3,4,6-hexa-O-galloyl-β-D-glucose, 1,2,3,4,6-hepta-O-galloyl-α-D-glucose, 1,2,3,4,6-hepta-O-galloyl-β-D-glucose, 1,2,3,4,6-octa-O-galloyl-α-D-glucose, 1,2,3,4,6-octa-O-galloyl-β-D-glucose, 1,2,3,4,6-nona-O-galloyl-α-D-glucose, 1,2,3,4,6-nona-O-galloyl-β-D-glucose, 1,2,3,4,6-deca-O-galloyl-α-D-glucose, and 1,2,3,4,6-deca-O-galloyl-β-D-glucose, or a salt theeof, and
wherein said gallotannin composition comprises less than 5% by dry weight of one or more of the following compounds: mono-O-galloyl-β-D-glucose, di-O-galloyl-β-D-glucose, tri-O-galloyl-β-D-glucose, tetra-O-galloyl-β-D-glucose, unadeca-O-galloyl-β-D-glucose, dodeca-O-galloyl-β-D-glucose or mixtures thereof.

14. The method of claim 13 wherein administration of said gallotannin composition inhibits differentiation of preadipocytes to adipocytes in the subject.

15. The method of claim 13 wherein said gallotannin composition is administered to the subject orally or by injection.

16. A method of inhibiting differentiation of preadipocytes to adipocytes in vitro or in vivo, comprising:
contacting the preadipocytes with a gallotannin composition,
wherein said gallotannin composition comprises one or more hydrolysable gallotannins selected from the group consisting of 1,2,3,4-tetra-O-galloyl-α-D-glucose, 1,2,3,6-tetra-O-galloyl-α-D-glucose, 1,3,4,6-tetra-O-galloyl-α-D-glucose, 1,2,3,4,6-penta-O-galloyl-α-D-glucose, 1,2,3,4,6-penta-O-galloyl-β-D-glucose, 1,2,3,4,6-hexa-galloyl-α-D-glucose, 1,2,3,4,6-hexa-O-galloyl-β-D-glucose, 1,2,3,4,6-hepta-O-galloyl-α-D-glucose, 1,2,3,4,6-hepta-O-galloyl-β-D-glucose, 1,2,3,4,6-octa-O-galloyl-α-D-glucose, 1,2,3,4,6-octa-O-galloyl-β-D-glucose, 1,2,3,4,6-nona-O-galloyl-α-D-glucose, 1,2,3,4,6-nona-O-galloyl-β-D-glucose, 1,2,3,4,6-deca-O-galloyl-α-D-glucose, and 1,2,3,4,6-deca-O-galloyl-β-D-glucose, or salts thereof.

17. The method of claim 16 wherein said gallotannin composition comprises less than 5% by dry weight of one or more of the following compounds: mono-O-galloyl-β-D-glucose, di-O-galloyl-β-D-glucose, tri-O-galloyl-β-D-glucose, tetra-O-galloyl-β-D-glucose, unadeca-O-galloyl-β-D-glucose, dodeca-O-galloyl-β-D-glucose or mixtures thereof.

18. The method of claim 16 wherein said gallotannin composition comprises at least 50% by dry weight of 1,2,3,4,6-penta-O-galloyl-α-D-glucose or 1,2,3,4,6-penta-O-galloyl-β-D-glucose or a combination of 1,2,3,4,6-penta-O-galloyl-α-D-glucose and 1,2,3,4,6-penta-O-galloyl-β-D-glucose.

19. The method of claim 16 wherein said gallotannin composition comprises at least 50% by dry weight of 1,2,3,4,6-penta-O-galloyl-β-D-glucose.

20. A method of inhibiting differentiation of preadipocytes to adipocytes in vitro or in vivo, comprising
contacting the preadipocytes with a composition comprising one or more compounds having the following structure, salts thereof:
R—X-A(ₙ)-X-A(_{q})-X-A(_{z}),
wherein R is selected from the group consisting of D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose, α-D-Xylose, α-D Lyxose, E-D Lyxose, α-D Arabinose, E-D Arabinose, α-D Ribose, E-D Ribose, D-Trehalose, D-Maltose, D-Cellobiose, myo-Inositol, D-glucitol,
X is an ester or ether linkage,
A is a trihydroxybenzoic acid selected from the group consisting of 3,4,5—trihydroxybenzoic acid, 2,3,4-trihydroxybenzoic acid 2,4,6-trihydroxybenzoic acid, or a dihydroxybenzoic acid selected from the group consisting of 2,3-dihydroxybenzoic acid, 2,4-dihydroxybenzoic acid, 3,4-dihydroxybenzoic acid, or a monohydroxybenzoic acids selected from the group consisting of 3-hydroxybenzoic acid and 4-hydroxybenzoic acid,
n is 5, q is 0, 1, 2, 3, 4, or 5, and z is 0 when R is D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose;
n is 4, q is 0, 1, 2, 3, or 4, and z is 0, 1, or 2 when R is (x-D-Xylose, α-D Lyxose, β-D Lyxose, α-D Arabinose, β-D Arabinose, α-D Ribose, β-D Ribose;
n is 6, q is 0, 1, 2, 3, 4, 5, or 6, and z is 0 when R is D-Glucitol or myo-Inositol, and
n is 8, q is 0, 1, 2, 3, 4, 5, 6, 7, or 8, and z is 0 when R is D-Trehalose, D-Maltose, or D-Cellobiose.

21. A gallotannin variant composition, comprising one or more gallotannin variant compounds, or a pharmaceutically acceptable salt thereof, wherein each of said gallotanin variant compounds have the following structure:
R—X-A(ₙ)-X-A(_{q})-X-A(_{z}),
wherein R is selected from the group consisting of D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose, α-D-Xylose, α-D Lyxose, β-D Lyxose, α-D Arabinose, β-D Arabinose, α-D Ribose, β-D Ribose, D-Trehalose, D-Maltose, D-Cellobiose, myo-Inositol, D-glucitol,
X is an ester or ether linkage,
A is a trihydroxybenzoic acid selected from the group consisting of 3,4,5-trihydroxybenzoic acid, 2,3,4-trihydroxybenzoic acid 2,4,6-trihydroxybenzoic acid, or a dihydroxybenzoic acid selected from the group consisting of 2,3-dihydroxybenzoic acid, 2,4-dihydroxybenzoic acid, 3,4-dihydroxybenzoic acid, or a monohydroxybenzoic acids selected from the group consisting of 3-hydroxybenzoic acid and 4-hydroxybenzoic acid,
n is 5, q is 0, 1, 2, 3, 4, or 5, and z is 0 when R is D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose;
n is 4, q is 0, 1, 2, 3, or 4, and z is 0, 1, or 2 when R is α-D-Xylose, α-D Lyxose, β-D Lyxose, α-D Arabinose, β-D Arabinose, α-D Ribose, β-D Ribose;
n is 6, q is 0, 1, 2, 3, 4, 5, or 6, and z is 0 when R is D-Glucitol or myo-Inositol, and
n is 8, q is 0, 1, 2, 3, 4, 5, 6, 7, or 8, and z is 0 when R is D-Trehalose, D-Maltose, or D-Cellobiose, and
wherein each of said gallotannin variant compounds is not a beta anomeric form of tetra-O-galloyl-D-glucose, penta-O-galloyl-D-glucose, hexa-galloyl-α-D-glucose, hepta-O-galloyl-D-glucose, octa-O-galloyl-D-glucose, nona-O-galloyl-D-glucose, or deca-O-galloyl-D-glucose.

22. The composition of claim 21 wherein the composition comprises Pentakis-O-(3,4-dihydroxybenzoyl)-β-D-glucopyranose, or tetrakis-O-(3,4,5-trihydroxybenzoyl)-β-D-xylopyranose.

23. A compound for treating diabetes insulin resistance, impaired glucose tolerance, hyperglycemia, hyperinsulinemia or obesity in a subject, said compound having the following formula:
R—X-A(ₙ)-X-A(_{q})-X-A(_{z}),
wherein R is selected from the group consisting of D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose, α-D-Xylose, α-D Lyxose, α-D Lyxose, α-D Arabinose, β-D Arabinose, α-D Ribose, β-D Ribose, D-Trehalose, D-Maltose, D-Cellobiose, myo-Inositol, D-glucitol,
X is an ester or ether linkage,
A is a trihydroxybenzoic acid selected from the group consisting of 3,4,5-trihydroxybenzoic acid, 2,3,4-trihydroxybenzoic acid 2,4,6-trihydroxybenzoic acid, or a dihydroxybenzoic acid selected from the group consisting of 2,3-dihydroxybenzoic acid, 2,4-dihydroxybenzoic acid, 3,4-dihydroxybenzoic acid, or a monohydroxybenzoic acids selected from the group consisting of 3-hydroxybenzoic acid and 4-hydroxybenzoic acid,
n is 5, q is 0, 1, 2, 3, 4, or 5, and z is 0 when R is D-Glucose, L-Glucose, D-Mannose, L-Mannose, D-Galactose, L-Galactose, D-Allose, L-Allose, D-Altrose, L-Altrose D-Gulose, L-Gulose, D-Idose, L-Idose, D-Talose, L, Talose, D-Fructose, L-Fructose;
n is 4, q is 0, 1, 2, 3, or 4, and z is 0, 1, or 2 when R is α-D-Xylose, α-D Lyxose, β-D Lyxose, α-D Arabinose, β-D Arabinose, α-D Ribose, β-D Ribose;
n is 6, q is 0, 1, 2, 3, 4, 5, or 6, and z is 0 when R is D-Glucitol or myo-Inositol, and
n is 8, q is 0, 1, 2, 3, 4, 5, 6, 7, or 8, and z is 0 when R is D-Trehalose, D-Maltose, or D-Cellobiose,
or a pharmaceutically acceptable salt of said compound.

24. A pharmaceutical composition for treating insulin resistance, impaired glucose tolerance, hyperglycemia, hyperinsulinemia or obesity in a subject, said pharmaceutical composition comprising an effective amount of the compound of claim 23 or a pharmaceutically acceptable salt thereof and a pharmaceutically acceptable carrier or diluent.
